(19) United States
(12) Patent Application Publication
(10) Pub. No.: US 2021/0348104 A1
(43) Pub. Date: Nov. 11, 2021

(54) MICROCOSM BIO-SCAFFOLD AND APPLICATIONS THEREOF

(71) Applicant: Volumetric Biotechnologies, Inc., Houston, TX (US)

(72) Inventors: Bagrat Grigoryan, Houston, TX (US); Jordan Miller, Houston, TX (US); Matthew Gelber, Houston, TX (US)

(21) Appl. No.: 17/307,880

(22) Filed: May 4, 2021

Related U.S. Application Data
(60) Provisional application No. 63/020,407, filed on May 5, 2020.

(51) Int. Cl. C12M 1/12; C12M 1/00; G01N 33/50; C12N 5/00

(52) U.S. Cl. CPC C12M 25/14; C12M 25/06; C12N 5/0018; G01N 33/5008; C12M 23/34

(57) ABSTRACT

A bio-assembly, a kit for a bio-assembly and methods of using the same are provided. The bio-assembly includes a substrate and a bio-scaffold affixed to the substrate. In accordance with various embodiments, a loader plate having a partition or a loader and a plate having a partition are provided. In accordance with various embodiments, the loader plate includes a partition outlet and a partition inlet, wherein the partition outlet and the partition inlet in fluid communication with the gel. In accordance with various embodiments, the partition comprising an internal volume and shaped to receive the bio-scaffold, and the loader includes a loader inlet and a loader outlet in fluid communication with the gel. In accordance with various embodiments, a bio-compatible adhesive positioned between the substrate and the loader plate or plate. In accordance with various embodiments, a fluid mixture is injected into the bio-scaffold.

### CROSS-REFERENCE TO RELATED APPLICATIONS

This application claims priority from U.S. Provisional Application No. 63/020,407, filed on May 5, 2020, and is incorporated by reference in their entirety.

### BACKGROUND

Pre-clinical research and drug development generally relies on testing the behavior of human cells in a flat petri dish and in animal models of human disease to understand physiology and predict the performance of drugs in the human body. These models can be overly simplistic and provide inadequate representations of the complex networked interactions actually taking place in the human body.

Currently, the majority of drug screening happens on flat plastic petri dishes or well plates. Mono-cultures of mouse, rat, or human cells are grown on these flat plastic surfaces and various drug candidates are added to these cultures and the behavior of cells is monitored over time. Thousands of compounds are screened this way and the most promising candidates are selected to progress towards animal testing. Typically, mice are selected as the animal model due to their relatively low cost, ease of handling, and ability to breed various strains of mice.

While screening in vitro on plastic plates is an important part of the process to distill drug candidates, the cellular environment on plastic plates is inaccurately reflected of the true cellular microenvironment. Cells on plastic plates are generally adhered to hard plastic material, where they attach, grow, and function in 2D, and are cultured under static conditions. Additionally, typically only one cell line is selected to grow on plates, even though the cellular microenvironment is comprised of various different cell types in close proximity. Therefore, improved tools and platforms that accurately reflect a more accurate cellular microenvironment are needed in cell culturing processes for various purposes including, for example, distilling drug candidates.

### SUMMARY

In accordance with various embodiments, a kit having a bio-assembly is provided. The bio-assembly includes a substrate, and a bio-scaffold affixed to the substrate; and a loader plate having a partition. The partition includes a partition outlet and a partition inlet, the partition outlet and the partition inlet in fluid communication with the bio-scaffold, and a bio-compatible adhesive positioned between the substrate and the loader plate, the adhesive configured to maintain a fluid-impermeable bond between the substrate and the loader plate.

In accordance with various embodiments, a kit having a bio-assembly is provided. The bio-assembly includes a substrate, and bio-scaffold affixed to the substrate. The plate includes partition having an internal volume and shaped to receive the bio-scaffold into the internal volume, and bio-compatible adhesive positioned between the substrate and the plate, the adhesive configured to maintain a bond between the substrate and the plate; loader having a loader inlet and a loader outlet, the loader inlet and loader outlet in fluid communication with the bio-scaffold; and a fluid mixture configured to be injected into the bio-scaffold.

In accordance with various embodiments, a method for generating a kit containing cells is provided. The method includes providing a bio-assembly comprising a substrate and a bio-scaffold affixed to the substrate, wherein the bio-scaffold comprises a vascular component having a vascular inlet and a vascular outlet; providing a loader plate comprising a partition comprising a partition outlet and a partition inlet; connecting the partition inlet to the vascular inlet and connecting the partition outlet to the vascular outlet; attaching cells to the vascular component; and perfusing the vascular component to form a cellular layer.

In accordance with various embodiments, a method for generating a cell culture is provided. The method includes providing a bio-assembly comprising a substrate and a bio-scaffold affixed to the substrate, wherein the bio-scaffold comprises a vascular component having a vascular inlet and a vascular outlet; providing a plate comprising a partition comprising an internal volume; providing a loader comprising a loader inlet and loader outlet; positioning the bio-scaffold with vascular component within the internal volume of the partition; connecting the loader inlet to the vascular inlet and connecting the loader outlet to the vascular outlet; attaching cells to the vascular component; and perfusing the vascular component to form a cellular layer.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a schematic diagram of an example bio-assembly kit, according to various embodiments.

FIG. 2 is a schematic diagram of another example bio-assembly kit, according to various embodiments.

FIG. 3A is a schematic illustration of an example bio-assembly kit, according to various embodiments.

FIG. 3B is another schematic illustration of the example bio-assembly kit of FIG. 3A.

FIGS. 3C and 3D are schematic illustrations of the example bio-assembly kit of FIGS. 3A and 3B, respectively, with a lid, in accordance with various embodiments.

FIG. 4A is a schematic illustration of an example bio-assembly kit, according to various embodiments.

FIG. 4B is a schematic illustration of another example bio-assembly kit, according to various embodiments.

FIG. 4C is a schematic illustration of another example bio-assembly kit, according to various embodiments.

FIG. 4D is a schematic illustration of another example bio-assembly kit, according to various embodiments.

FIG. 4E is a schematic illustration of another example bio-assembly kit, according to various embodiments.

FIG. 5A is a schematic illustration of an example bio-assembly kit, according to various embodiments.

FIG. 5B is another schematic illustration of the example bio-assembly kit of FIG. 5A.

FIGS. 6A, 6B, 6C illustrate various stages of cell loading in an example bio-assembly, according to various embodiments.

FIGS. 7A, 7B, 7C illustrate various stages of cell loading in an example bio-assembly having an array of bio-scaffolds, according to various embodiments.

FIG. 8 is a flowchart for a method of generating a cell culture, in accordance with various embodiments.

FIG. 9 is another flowchart for a method of generating a cell culture, in accordance with various embodiments.

### DETAILED DESCRIPTION

Currently, many have been exploring migration assays which involves use of permeable, polymer-based Transwell supports. These microporous supports are placed on top of a well plate containing media, cells are added into these supports and allowed to grow. Compounds are then added into the support that contains a flat monolayer of cells and the ability of the compound to increase the vascular permeability is evaluated by measuring the concentration of the compound that passes through the Transwell into the well. In some cases, the compounds will increase vascular permeability. In some cases, the compounds are to be tested that it passes through the cell layer into the second compartment. The Transwell is a tool for other readouts in addition to vascular permeability, such as, cell migration, promoting air-liquid interface, etc. While this assay is commonly used for studying vascular permeability, it doesn't properly mimic in vivo compound transport behavior due to the limited nature of the cellular environment which lack key features such as the presence of multiple cell types, constant perfusion and chemical and mechanical characteristics of the extracellular matrix.

While testing the safety and efficacy of drug compounds in small animal models such as mice is an important step in evaluating the overall safety and efficacy of the compounds, mice are non-ideal models for developing human therapies because mice do not represent human anatomy or physiology. Thousands of compounds have been shown to be effective in mice but the results don't transfer over during human clinical trials, where the majority of drugs fail in Phase II. Additionally, there is significant variability from mouse to mouse, and even the way that mice are handled during experiments has been shown to dramatically influence outcome.

In accordance with various embodiments, various technologies, platforms, and methods for cell culturing are described herein. In accordance with various embodiments, the disclosed platforms, templates, configurations, and implementations offer a truer cellular microenvironment that can improve cell culturing processes for distilling drug candidates. In accordance with various embodiments, the disclosed platform, also referred to herein as a bio-scaffold, contains features that mimic human anatomy and physiology, resulting in biomimetic human tissue models resulting in better human data obtained towards understanding the safety and efficacy of drug candidates. In accordance with various embodiments, the disclosed bio-scaffold can comprise cell-adhesive and cell-degradable materials. In accordance with various embodiments, the bioactive scaffold includes cell-adhesive and cell-degradable materials where cells can adhere, grow, and migrate onto a matrix they can remodel over time by secreting matrix metalloproteinases (MMPs) and depositing their own extracellular matrix (ECM).

In accordance with various embodiments, the bio-scaffold can contain a vascular component, which enables cells to be under perfusion conditions. In accordance with various embodiments, more than one vascular component can be incorporated into the same bio-scaffold volume. In accordance with various embodiments, fluids including gases and liquids, such as media or blood, can be introduced into the vascular components. In accordance with various embodiments, the bio-scaffold can contain empty chambers onto which cells or other biological material can be introduced. In accordance with various embodiments, a vascular component can be defined as a bounded void volume topology that is suitable for flow of fluids including liquids and gases.

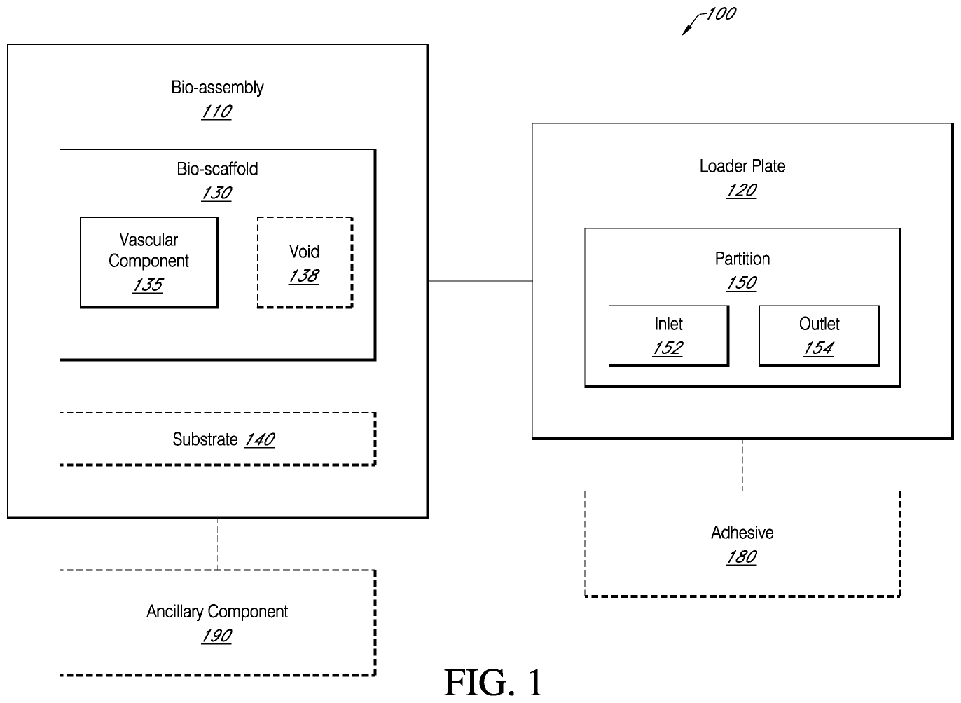

In accordance with various embodiments, the chambers that include the bio-scaffold is secured to contain inlet and outlet connections to be connected to perfusion via syringe pump, peristaltic pump, pneumatic pump, or gravity driven flow, or connected to the blood supply of an animal. In accordance with various embodiments, these inlets and outlets can be placed on any side of the chambers, depending on the architecture of the vascularized bio-scaffold. In accordance with various embodiments, the bio-assembly can be combined with a loading apparatus (or a loader or loader plate) to enable perfusing in the vascular component of the bio-scaffold. In accordance with various embodiments, the bio-assembly and the loader can be combined along with various ancillary components to form a bio-assembly kit. In accordance with various embodiments, multiple bio-scaffolds can be placed within the chambers, enabling for arraying of bio-scaffolds for high-throughput experimentation. In accordance with various embodiments, the bio-scaffold can be configured to serve as a mini-organ and may be transplanted for therapeutic use. In accordance with various embodiments, each of the array of bio-scaffolds can be configured to serve as a mini-organ that is different from another in the array of bio-scaffolds, and may be transplanted for therapeutic use. In accordance with various embodiments, each of the array of bio-scaffolds can be individually and independently pumped at a flow rate of fluid that is different from another of the array of bio-scaffolds. In accordance with various embodiments, each of the array of bio-scaffolds can be pumped with a different fluid or fluid mixture that is individually tailored, and different from another of the array of bio-scaffolds. Various configurations, embodiments, and implementations of the technologies, platforms, and methods for cell culturing are described in further detail with respect to FIGS. 1-9. In accordance with various embodiments, various configurations, embodiments, and implementations of the technologies, platforms, and methods disclosed herein for cell culturing can be applicable to any of the example embodiments and configurations described and presented with respect to the following FIGS. 1-9.

Referring now to FIG. 1, which is a schematic diagram of a bio-assembly kit 100, according to various embodiments. In accordance with various embodiments, the bio-assembly kit 100 includes a bio-assembly 110, a loader plate 120, and can optionally include adhesive 180 and/or ancillary component 190. In accordance with various embodiments, the bio-assembly 110 includes a bio-scaffold 130. In accordance with various embodiments, the bio-assembly 110 optionally includes a substrate 140. In accordance with various embodiments, the bio-scaffold 130 includes a vascular component 135. In accordance with various embodiments, the bio-scaffold 130 optionally includes a void 138. In accordance with various embodiments, the loader plate 120 includes a partition 150. In accordance with various embodiments, the partition 150 can include a partition inlet 152 and a partition outlet 154.

In accordance with various embodiments, the bio-assembly 110 includes the bio-scaffold 130 affixed or otherwise disposed on the substrate 140. In accordance with various embodiments, the bio-scaffold 130 is affixed or otherwise disposed on the substrate 140 via any suitable bonding techniques, including for example, but not limited to, covalently bonding the bio-scaffold 130 to a top surface of the substrate 140, which can be either functionalized with silane or any other means to promote adhesion between the bio-scaffold 130 and the substrate 140. In accordance with various embodiments, adhesive can include tape, liquid adhesive/glue, or UV curable materials, or any other suitable materials. In accordance with various embodiments, a substrate is a glass slide that is intimately in contact with the partition. In accordance with various embodiments, the bio-scaffold 130 is a hydrogel that can be disposed on the substrate 140 without covalent bonding. In accordance with various embodiments, the bio-scaffold 130 can be disposed on the substrate 140.

In accordance with various embodiments, the substrate 140 can be used as a substrate in cell culturing environments. In accordance with various embodiments, the substrate 140 can be transparent glass or plastics, or any other suitable material, such as for example, but not limited to polycarbonate, polysulfone, polymethyl methacrylate, polystyrene, cyclic olefin copolymer, polyethylene, polypropylene, glass, quartz, mica, infrared-transparent salts, such as calcium bromide, potassium bromide, or any of these materials combined with a thin film of any other material, or with a thin metallic film to enable surface plasmon based measurements.

In accordance with various embodiments, the bio-scaffold 130 can be a gel, a hydrogel, polymerizable hydrogel including, for example, water and poly(ethylene glycol) diacrylate (PEGDA) having 6 kDa, 20 weight %, lithium acylphosphinate (LAP) which absorbs in the ultraviolet to visible light wavelength range, gelatin methacrylate, or any other suitable hydrogel materials, including but not limited to any of collagen methacrylate, silk methacrylate, hyaluronic acid methacrylate, chondroitin sulfate methacrylate, elastin methacrylate, cellulose acrylate, dextran methacrylate, heparin methacrylate, NIPAAm methacrylate, Chitosan methacrylate, polyethylene glycol norbornene, polyethylene glycol dithiol, thiolated gelatin, thiolated chitosan, thiolated silk, PEG based peptide conjugates, or any combination thereof. In accordance with various embodiments, the bio-scaffold 130 can include any material, including those listed above, that is 3D printable or moldable, including for example, via injection molding techniques, rapid casting or sacrificial molding. In accordance with various embodiments, the bio-scaffold 130 can be formed via casting around a pattern, such as a needle or a structure, which can be removed mechanically, chemically, and/or by light-induced degradation. In accordance with various embodiments, the bio-scaffold 130 can be formed via casting around a pattern that can be removed mechanically, chemically, or by light-induced degradation, followed by patterning one or more pieces and then bonding the pieces together.

In accordance with various embodiments, the bio-scaffold 130 are perfusable hydrogels. In accordance with various embodiments, the bio-assembly 110 can include a hydrophilic component and a hydrophobic component. In accordance with various embodiments, the bio-scaffold 130 can include aqueous pre-hydrogel solutions containing organic materials, such as for example, tartrazine (yellow food coloring FD&C Yellow 5, E102), curcumin (from turmeric), or anthocyanin (from blueberries) each of which can yield hydrogels, and inorganic gold nanoparticles having a diameter, for example, from about 5 nm to 100 nm, for biocompatibility and light attenuating properties, and for functionalities for acting as, for example, an effective photoabsorbing additive to generate perfusable hydrogels. In accordance with various embodiments, the bio-scaffold 130 can include a photoabsorber. In accordance with various embodiments, the photoabsorber can be hydrophilic. In accordance with various embodiments, the hydrophilic photoabsorber can be one of a food dye, tartrazine, Sunset Yellow FCF (Yellow No. 6), Brilliant Blue FCF (FD&C Blue No. 1), indigo carmine (FD&C Blue No. 2), Fast Green FCF (FD&C Green No. 3) anthocyanins, anthocyanidin, erythrosine (FD&C Red No. 3), Allura Red AC (FD&C Red No. 40), riboflavin (Vitamin B2, E101, E101a, E106), ascorbic acid (vitamin C), Quinoline Yellow WS, carmoisine (azorubine), Ponceau 4R (E124), Patent Blue V (E131), Green S (E142), Yellow 2G (E107), Orange GGN (E111), Red 2G (E128), caramel color, phenol red, methyl orange, 4-nitrophenol, NADH disodium salt, or any combination thereof. In accordance with various embodiments, the photoabsorber can be hydrophobic. In accordance with various embodiments, the hydrophobic photoabsorber can be one of curcumin (E100), turmeric, alpha carotene, beta carotene, canthaxanthin (keto-carotenoid), cochineal extract, paprika, saffron, ergocalciferol (vitamin D2), cholecalciferol (vitamin D3), Citrus Red 2, annatto extract, Lycopene, or any combination thereof.

In accordance with various embodiments, the bio-scaffold 130 includes one or more vascular components 135. In accordance with various embodiments, the bio-scaffold 130 along with the one or more vascular components 135 can be 3D printed or molded. In accordance with various embodiments, the one or more vascular component 135 includes a vascular inlet and a vascular outlet. In accordance with various embodiments, the one or more vascular components 135 includes one or more channels that may branch out as a tree-like structure within the bio-scaffold 130. In accordance with various embodiments, the one or more channels of the one or more vascular components 135 may include branches that can form, for example, as a torus knot, wherein the channels re-converge at another point within the bio-scaffold 130. In accordance with various embodiments, the one or more vascular components 135 can include branched structures that can extend from various portions of the bio-scaffold 130 and terminate at other portions within the bio-scaffold 130. In accordance with various embodiments, the one or more vascular components 135 can have a multiscale vasculature having branches and taperings similar to that of organs in human body.

In accordance with various embodiments, the one or more vascular components 135 have one or more channels of any shape in cross-section or aspect ratios that have a cross-section dimension or width (e.g., the cross-section dimension is a diameter if circular) ranging from about 10 μm to about 1 mm, 100 μm to about 500 μm, or about 800 microns or less, about 500 microns or less, about 400 microns or less, about 300 microns or less, or 200 microns or less. In accordance with various embodiments, the one or more vascular components 135 are perfusable. In accordance with various embodiments, the one or more channels of the one or more vascular components 135 are expandable in response to increases in pressure, mechanical, electrical, and/or chemical stimuli within the one or more vascular components 135. In accordance with various embodiments, the one or more channels of the one or more vascular components 135 are contractable in response to pressure, mechanical, electrical, and/or chemical stimuli within the one or more vascular components 135.

In accordance with various embodiments, the one or more vascular components 135 can include a narrowing inlet and a narrowing outlet. In accordance with various embodiments, the one or more vascular components 135 can include one or more vascular inlets. In accordance with various embodiments, the one or more vascular components 135 can include one or more vascular outlets. In accordance with various embodiments, each of the one or more vascular components 135 can include a vascular inlet and a vascular outlet. In accordance with various embodiments, the vascular inlet and vascular outlet for a first vascular component of the one or more vascular components 135 is positioned orthogonal or substantially orthogonal, parallel or substantially parallel, or at an angle between 0 degree and 90 degrees, with respect to the vascular inlet and vascular outlet of a second vascular component of the one or more vascular components 135.

In accordance with various embodiments, each of the one or more vascular components 135 can include a chamber or compartment in the bio-assembly 110 where a flowable suspension of cells is injected. In accordance with various embodiments, each of the one or more vascular components 135 can include different chambers or different compartment types in the bio-assembly 110 where different cell types are injected into different compartments.

In accordance with various embodiments, the bio-scaffold 130 optionally includes the void 138. In accordance with various embodiments, the one or more vascular components 135 is disposed in the void 138. In accordance with various embodiments, the bio-scaffold 130 includes one or more voids 138.

In accordance with various embodiments, the loader plate 120 includes the partition 150 which includes the partition inlet 152 and the partition outlet 154. In accordance with various embodiments, the partition inlet 152 and the partition outlet 154 are substantially parallel to a top surface of the loader plate 120. In accordance with various embodiments, the partition inlet 152 and the partition outlet 154 are adjacent to each other and are disposed on the same side of the loader plate 120. In accordance with various embodiments, the partition inlet 152 and the partition outlet 154 are disposed on the different sides of the loader plate 120. In accordance with various embodiments, the partition inlet 152 and the partition outlet 154 are disposed on the opposite sides of the loader plate 120. In accordance with various embodiments, the partition inlet 152 and the partition outlet 154 have tapering or gradually tapering tips.

In accordance with various embodiments, the loader plate 120 includes a material including, but not limited to resin, dental resin, biocompatible resin, clear polycarbonate, clear acrylic, transparent glass or plastics, or any other suitable material, such as for example, but not limited to polycarbonate, polysulfone, polymethyl methacrylate, polystyrene, cyclic olefin copolymer, polyethylene, polypropylene, glass, quartz, mica, infrared-transparent salts, such as calcium bromide, potassium bromide, or any combination thereof.

In accordance with various embodiments, the loader plate 120 has a lateral dimension (e.g., X or Y direction) ranging between 1 mm and 1,000 mm. In accordance with various embodiments, the loader plate 120 is between 1 mm to 1,000 mm in a first direction (e.g., X direction) and between 1 mm to 1,000 mm in a second direction (e.g., X direction). In accordance with various embodiments, the loader plate 120 has a dimension (e.g., X direction and Y direction) of 1 mm×1 mm, 1 mm×10 mm, 1 mm×100 mm, 1 mm×1,000 mm, 10 mm×1 mm, 10 mm×10 mm, 10 mm×100 mm, 10 mm×1,000 mm, 100 mm×1 mm, 100 mm×10 mm, 100 mm×100 mm, 100 mm×1,000 mm, 1,000 mm×1 mm, 1,000 mm×10 mm, 1,000 mm×100 mm, 1,000 mm×1,000 mm, 130 mm×90 mm, 90 mm×130 mm, or inclusive of any lateral dimension, including any incremental integer or decimal values, thereof.

In accordance with various embodiments, the partition 150 has a lateral dimension that is between 0.1% to 10%, 10% to 20%, 20% to 30%, 30% to 40%, 40% to 50%, 50% to 60%, 60% to 70%, 70% to 80%, 80% to 90%, 90% to 99.9%, or inclusive of any lateral dimension, including any incremental integer or decimal values, thereof, of the loader plate 120 in both X and Y directions. In accordance with various embodiments, the partition 150 has a lateral dimension that is between 0.1 mm and 999 mm. In accordance with various embodiments, the partition 150 is between 0.1 to 100 mm, 100 to 200 mm, 200 to 300 mm, 300 to 400 mm, 400 to 500 mm, 500 to 600 mm, 600 to 700 mm, 700 to 800 mm, 800 to 900 mm, 900 to 999 mm, or inclusive of any X value, including any incremental integer or decimal values in the X direction; and between 0.1 to 100 mm, 100 to 200 mm, 200 to 300 mm, 300 to 400 mm, 400 to 500 mm, 500 to 600 mm, 600 to 700 mm, 700 to 800 mm, 800 to 900 mm, 900 to 999 mm, or inclusive of any Y value, including any incremental integer or decimal values, thereof in the Y direction.

In accordance with various embodiments, the loader plate 120 includes a plurality of partitions 150. In accordance with various embodiments, each of the plurality of partitions 150 includes a partition inlet 152 and the partition outlet 154. In accordance with various embodiments, the loader plate 120 includes 1 to 1000 partitions, including 3, 4, 6, 12, 24, 48, 96, 192 or 384 partitions 150. In accordance with various embodiments, each of the plurality of partitions 150 includes at least one set, and at most 20 sets, of partition inlets 152 and partition outlets 154.

In accordance with various embodiments, the loader plate 120 includes the partition 150 that includes an internal volume. In accordance with various embodiments, each of the plurality of partitions 150 includes an inner volume. In accordance with various embodiments, each of the plurality of partitions 150 is shaped to receive the bio-assembly 110 into the internal volume.

In accordance with various embodiments, the partition inlet 152 and the partition outlet 154 are in fluid communication with the bio-scaffold 130. In accordance with various embodiments, the vascular inlet is in fluid communication with the partition inlet 152. In accordance with various embodiments, the vascular outlet is in fluid communication with the partition outlet 154. In accordance with various embodiments, each of the one or more vascular inlets is in fluid communication with an associated partition inlet and each of the one or more vascular outlets is in fluid communication with an associated partition outlet.

In accordance with various embodiments, the bio-assembly 110 and partition fluid communication is mediated by a tapered constriction in the bio-assembly 110 and provides a fluidic seal at a normal operating fluid pressure during perfusion. In accordance with various embodiments, the fluidic seal is provided size differential between a larger size of the inlet 152/outlet 154 of the partition 150 and smaller size of the inlet/outlet of the bio-scaffold 130. In accordance with various embodiments, the mechanical fit between the female (inlet/outlet of the bio-scaffold 130) and the male (the inlet 152/outlet 154 of the partition 150) provides an “interference fit” between the male and female features. In accordance with various embodiments, an adapter may be used to provide a fluidic seal between the inlet 152/outlet 154 of the partition 150 and the inlet/outlet of the bio-scaffold 130.

In accordance with various embodiments, perfusing can occur under various perfusing mechanisms, such as for example, but not limited to under gravity flow, via a pump for positive pressure or via vacuum suctioning for negative pressure. In accordance with various embodiments, the normal operating fluid pressure at the inlets and outlets is between about −100 kPa (negative pressure, such as, suction) and about 100 kPa (positive pressure, such as, pumped fluid, liquid or gas), between about −50 kPa and about 50 kPa, between about −15 kPa and about 15 kPa, between about −10 kPa and about 10 kPa, between about −1 kPa and about 1 kPa, or between about −0.1 kPa and about 0.1 kPa, inclusive of any ranges therebetween. In accordance with various embodiments, the normal operating fluid pressure is between about 1 Pa and about 100 kPa, between about 1 Pa and about 50 kPa, between about 1 Pa and about 15 kPa, between about 1 Pa and about 10 kPa, between about 1 Pa and about 1 kPa, or between about 1 Pa and about 0.1 kPa, inclusive of any ranges therebetween. In accordance with various embodiments, the normal operating fluid pressure is between about −100 kPa and about −1 Pa, between about −50 kPa and about −1 Pa, between about −15 kPa and about −1 Pa, between about −10 kPa and about −1 Pa, between about −1 kPa and about −1 Pa, or between about −0.1 kPa and about −1 Pa, inclusive of any ranges therebetween.

In accordance with various embodiments, perfusing can occur at a fluid flow rate that does not shear cells that line the vasculature of the bio-scaffold 130. In accordance with various embodiments, perfusing can occur at a flow rate between about 1 nL/min to about 100 mL/min, about 10 nL/min to about 10 mL/min, about 100 nL/min to about 1 mL/min, about 1 μL/min to about 1 mL/min, about 1 μL/min to about 100 μL/min, or about 10 μL/min to about 100 μL/min, about 1 mL/min to about 100 mL/min inclusive of any ranges therebetween. In accordance with various embodiments, perfusing can occur to mimic tidal ventilation, that may include positive pressure perfusion, with variations on flow like blood pumping (e.g., heart beat mimic), or continuous flow or within a flow regime without shearing the bio-scaffold b130. For example, perfusing can be performed with high-glucose media for about 3 hours, about 6 hours, about 9 hours, or about 12 hours followed by low glucose media for about 3 hours, about 6 hours, about 9 hours, or about 12 hours, mimicking when a person eats a meal.

In accordance with various embodiments, the bio-scaffold kit 100 optionally includes the adhesive 180. In accordance with various embodiments, the bio-scaffold kit 100 optionally includes a clamping tool or mechanism to maintain adhesion. In accordance with various embodiments, the adhesive 180 is a bio-compatible and/or cytocompatible adhesive positioned between the substrate 140 and the loader plate 120. In accordance with various embodiments, the adhesive 180 is configured to maintain a fluid-impermeable bond between the substrate 140 and the loader plate 120. In accordance with various embodiments, the adhesive 180 includes a degradable or bio-degradable material. In accordance with various embodiments, the adhesive 180 includes a material such as, but limited to a liquid adhesive, such as a 2-part epoxy, a photo-activated epoxy, or a cyanoacrylate, or a tape adhesive, such as an acrylic tape adhesive, such as 3M LSE9474.

In accordance with various embodiments, the adhesive 280 has a lateral dimension similar to that of the loader plate 120 and a thickness ranging between 0.1 μm and 5 mm, including between 0.1 μm and 1 μm, 1 μm and 10 μm, 10 μm and 100 μm, 100 μm and 1 mm, or 1 mm and 5 mm, inclusive of any thickness values therebetween.

In accordance with various embodiments, the bio-scaffold kit 100 optionally includes the ancillary component 190. In accordance with various embodiments, the ancillary component 190 can include any material that can flow within the one or more vascular component 135 or inside the void 138 of the bio-assembly 110. In accordance with various embodiments, the ancillary component 190 can include a fluid mixture having multiple fluid components. In accordance with various embodiments, the ancillary component 190 can include a fluid mixture that includes a liquid, foam, or secondary pre-matrix. In accordance with various embodiments, the ancillary component 190 can include a fluid mixture that can be injected into the bio-assembly 110.

In accordance with various embodiments, the void 138 of the bio-scaffold 130 of the bio-assembly 110 is a perfusable or injectable space with one or more inlets. In accordance with various embodiments, the void 138 of the bio-scaffold 130 of the bio-assembly 110 is a perfusable or injectable space with one or more outlets. Alternatively, and in accordance with various embodiments, the void 138 can have no inlets or outlets. In accordance with various embodiments, the ancillary component 190, such as the fluid mixture, can be configured to be combined with live cells, the combination being injectable into the void 138. In accordance with various embodiments, the void 138 includes physical anchors that the ancillary component 190, such as the fluid mixture is dispensed around.

In accordance with various embodiments, the ancillary component 190 includes perfusable media, such as for example, complete media, with oxygen carriers, with red blood cells, whole human blood, and/or de-fibrinated human blood that does not clot. In accordance with various embodiments, the ancillary component 190 can include a fluid or liquid, such as for example, but not limited to bile, blood, urine, lymph, and/or a gas within the one or more vascular component 135 or inside the void 138 of the bio-assembly 110. In accordance with various embodiments, the ancillary component 190 can include a material that can form parenchymal tissue, such as for example, but not limited to liver, kidney, pancreas, lung, heart, interstitial tissue, such as for example, but not limited to fibroblasts, mesenchymal stem cells (MSCs) and other matrix producing and support cells. In accordance with various embodiments, the ancillary component 190 can include a material that can form a “clean meat”, such as those that appear similar to a marbled structure of Kobe beef.

In accordance with various embodiments, the ancillary component 190 can include cells or cell types that can form one or more layers from the list consisting of an endothelial layer, an epithelial layer, a smooth muscle cell layer, a sequentially delivered smooth muscle cell layer and endothelial layer, a sequentially delivered smooth muscle cell layer and epithelial layer, a sequentially delivered smooth muscle cell layer, gel layer, endothelial or epithelial layer, a sequentially delivered pericyte layer and endothelial layer, or a sequentially delivered pericyte layer and epithelial layer.

In accordance with various embodiments, the bio-scaffold kit 100 can include a bio-scaffold 130 having one or more vascular components 135 that already include cells or cell types (e.g., already lined with) from the list consisting of an endothelial layer, an epithelial layer, a smooth muscle cell layer, a sequentially delivered smooth muscle cell layer and endothelial layer, a sequentially delivered smooth muscle cell layer and epithelial layer, a sequentially delivered smooth muscle cell layer, gel layer, endothelial or epithelial layer, a sequentially delivered pericyte layer and endothelial layer, or a sequentially delivered pericyte layer and epithelial layer.

In accordance with various embodiments, the ancillary component 190 includes 3D printable materials, such as for example, but not limited to interstitial cells, such as fibroblasts, hMSCs and endothelial cells within the bio-scaffold 130 of the bio-assembly 110. In accordance with various embodiments, the ancillary component 190 includes biological matrix materials, such as for example, but not limited to fibrinogen, methacrylated fibrinogen, matrigel, collagen methacrylate, silk methacrylate, hyaluronic acid methacrylate, chondroitin sulfate methacrylate, elastin methacrylate, cellulose acrylate, dextran methacrylate, heparin methacrylate, NIPAAm methacrylate, chitosan methacrylate, polyethylene glycol norbornene, polyethylene glycol dithiol, thiolated gelatin, thiolated chitosan, thiolated silk, PEG based peptide conjugates, or any combination thereof.

In accordance with various embodiments, the ancillary component 190 can be included in the bio-scaffold kit 100 and cryopreserved at a temperature, for example, below 10° C., 0° C., −10° C., −25° C., −50° C., −75° C., −100° C., −150° C., −200° C., −250° C. or −270° C.

In accordance with various embodiments, a bio-scaffold system includes the bio-assembly 110 that can be tailored to adapt to tissue types by adding particular cells or ECM. In accordance with various embodiments, the bio-assembly 110 includes a blank bio-scaffold with architecture, whereby injected cells of the ancillary component 190 can take up residence, proliferate, migrate, invade the vasculature like metastasis. In accordance with various embodiments, the bio-scaffold system can be tailored under the control of exogenous factors similar to chemotherapeutics. In accordance with various embodiments, the bio-assembly 110 includes a blank bio-scaffold with architecture that can be injected with any type of cells to obtain an assay having a cell culture of that particular injected cell type. In accordance with various embodiments, the bio-assembly 110 includes a blank bio-scaffold with architecture that is injected with cancer cells to obtain a cancer invasion assay. In accordance with various embodiments, the bio-assembly 110 includes a blank bio-scaffold with architecture that is injected with liver cells to obtain a liver toxicology screening platform. In accordance with various embodiments, the bio-assembly 110 includes a blank bio-scaffold with architecture that is injected with cardiac cells to obtain a cardiac toxicology screening platform. In accordance with various embodiments, the bio-assembly 110 includes a blank bio-scaffold with architecture that is injected with kidney cells to obtain a kidney toxicology screening platform. In accordance with various embodiments, the bio-assembly 110 includes a blank bio-scaffold with architecture that is injected with brain cells to obtain a brain toxicology screening platform. In accordance with various embodiments, the bio-assembly 110 includes a blank bio-scaffold with architecture that is injected with gut cells to obtain a gut toxicology or gut permeability screening platform. In accordance with various embodiments, the bio-assembly 110 includes a blank bio-scaffold with architecture that is injected with lung cells to obtain a lung toxicology or gas transport screening platform. In accordance with various embodiments, the bio-assembly 110 offers identical tissue architecture suitable for high reproducibility and high throughput screening.

In accordance with various embodiments, the bio-scaffold system includes a plurality of bio-assemblies 110, where each of the plurality of bio-assemblies 110 can be tailored to adapt to different tissue types by adding particular cells or ECM. In accordance with various embodiments, the bio-scaffold system includes a plurality of bio-assemblies 110, where each of the plurality of bio-assemblies 110 can be tailored to include the same tissue type by adding particular cells or ECM.

In accordance with various embodiments, each of the plurality of bio-assemblies 110 can be perfused at a single fluid flow rate for each of the bio-assemblies 110, enabling for arraying of bio-assemblies for high-throughput experimentation. In accordance with various embodiments, each of the plurality of bio-assemblies 110 can be perfused at a different fluid flow rate and/or different pressure, independently, for each of the bio-assemblies 110. That is, each of the plurality of bio-assemblies 110 can be individually and independently pumped at a flow rate of fluid or with a different combination of pressures at the inlet and outlet that is different from another of the plurality of bio-assemblies 110.

In accordance with various embodiments, each of the plurality of bio-assemblies 110 can be pumped with the same fluid or fluid mixture for perfusing the plurality of bio-assemblies 110. In accordance with various embodiments, each of the plurality of bio-assemblies 110 can be pumped with a different fluid or fluid mixture that is individually tailored, and different from another of the plurality of bio-assemblies 110. In accordance with various embodiments, each of the plurality of bio-assemblies 110 can be configured to serve as a mini-organ and may be transplanted for therapeutic use. In accordance with various embodiments, each of the plurality of bio-assemblies 110 can be configured to serve as a mini-organ that is different from another in the plurality of bio-assemblies 110, and may be transplanted for therapeutic use.

In accordance with various embodiments, the bio-assembly 110 is substantially transparent. In accordance with various embodiments, the bio-assembly 110 is transparent and suitable for imaging with visible light, fluorescence, and/or luminescence. In accordance with various embodiments, the bio-assembly 110 is transparent and suitable for imaging after histological, immunohistochemistry, or immunofluorescence staining following sectioning via a vibratome, microtome, or a cryostat machine. In accordance with various embodiments, the bio-assembly 110 includes regions that are non-cellularized regions that provide optical conduits for imaging.

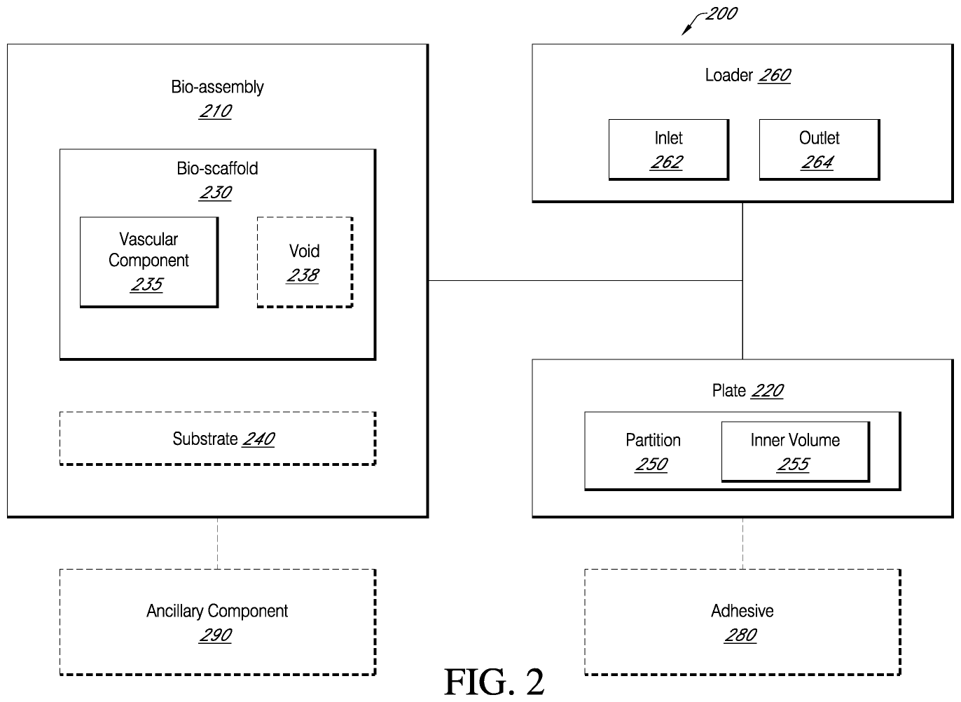

FIG. 2 is a schematic diagram of a bio-scaffold kit 200, according to various embodiments. In accordance with various embodiments, the bio-scaffold kit 200 includes a bio-assembly 210, a plate 220, a loader 260 and can optionally include adhesive 280 and/or ancillary component 290. In accordance with various embodiments, the bio-assembly 210 includes a bio-scaffold 230. In accordance with various embodiments, the bio-assembly 210 optionally includes a substrate 240. In accordance with various embodiments, the bio-scaffold 230 includes a vascular component 235. In accordance with various embodiments, the bio-scaffold 230 optionally includes a void 238. In accordance with various embodiments, the plate 220 includes a partition 250. In accordance with various embodiments, the loader 260 can include a loader inlet 262 and a loader outlet 264.

In accordance with various embodiments, the bio-assembly 210 includes the bio-scaffold 230 affixed or otherwise disposed on the substrate 240. In accordance with various embodiments, the bio-scaffold 230 is affixed or otherwise disposed on the substrate 240 via any suitable bonding techniques, including for example, but not limited to, covalently bonding the bio-scaffold 230 to a top surface of the substrate 240, which can be either functionalized with silane or any other means to promote adhesion between the bio-scaffold 230 and the substrate 240. In accordance with various embodiments, adhesive can include tape, liquid adhesive/glue, or UV curable materials, or any other suitable materials. In accordance with various embodiments, a substrate is a glass slide that is intimately in contact with the partition. In accordance with various embodiments, the bio-scaffold 230 is a hydrogel that can be disposed on the substrate 240 without covalent bonding. In accordance with various embodiments, the bio-scaffold 230 can be disposed on the substrate 240.

In accordance with various embodiments, the substrate 240 includes identical material as the substrate 140, and therefore will not be described in further detail.

In accordance with various embodiments, the bio-scaffold 230 includes identical material as the bio-scaffold 130, and therefore will not be described in further detail.

In accordance with various embodiments, the bio-scaffold 230 includes one or more vascular components 235. In accordance with various embodiments, the bio-scaffold 230 along with the one or more vascular components 235 can be 3D printed or molded. In accordance with various embodiments, the one or more vascular component 235 includes a vascular inlet and a vascular outlet. In accordance with various embodiments, the one or more vascular components 235 includes one or more channels that may branch out within the bio-scaffold 230. In accordance with various embodiments, the one or more channels of the one or more vascular components 235 may include branches that can form, for example, as a torus knot, wherein the channels re-converge at another point within the bio-scaffold 230. In accordance with various embodiments, the one or more vascular components 235 can include branched structures that can extend from various portions of the bio-scaffold 230 and terminate at other portions within the bio-scaffold 230. In accordance with various embodiments, the one or more vascular components 235 can have a multiscale vasculature having branches and taperings similar to that of organs in human body.

In accordance with various embodiments, the one or more vascular components 235 have one or more channels of any shape in cross-section or aspect ratios that have a cross-section dimension or width (e.g., the cross-section dimension is a diameter if circular) ranging from about 5 μm to about 5 mm, about 10 μm to about 3 mm, about 10 μm to about 1 mm, about 20 μm to about 500 μm, about 50 μm to about 500 μm, about 50 μm to about 1 mm, or about 50 μm to about 3 mm, inclusive of any ranges therebetween.

In accordance with various embodiments, the one or more vascular components 235 have one or more channels of any shape in cross-section or aspect ratios that have a cross-section dimension or width (e.g., the cross-section dimension is a diameter if circular) of about 800 μm or less, about 500 μm or less, about 400 μm or less, about 300 μm or less, about 200 μm or less, about 100 μm or less, or about 50 μm or less. In accordance with various embodiments, the one or more vascular components 235 are perfusable. In accordance with various embodiments, the one or more channels of the one or more vascular components 235 are expandable in response to increases in pressure, mechanical, electrical, and/or chemical stimuli within the one or more vascular components 235. In accordance with various embodiments, the one or more channels of the one or more vascular components 235 are contractable in response to pressure, mechanical, electrical, and/or chemical stimuli within the one or more vascular components 235.

In accordance with various embodiments, the one or more vascular components 235 can include a narrowing inlet and a narrowing outlet. In accordance with various embodiments, the one or more vascular components 235 can include one or more vascular inlets. In accordance with various embodiments, the one or more vascular components 235 can include one or more vascular outlets. In accordance with various embodiments, each of the one or more vascular components 235 can include a vascular inlet and a vascular outlet. In accordance with various embodiments, the vascular inlet and vascular outlet for a first vascular component of the one or more vascular components 235 is positioned substantially orthogonal to the vascular inlet and vascular outlet of a second vascular component of the one or more vascular components 235.

In accordance with various embodiments, each of the one or more vascular components 235 can include a chamber or compartment in the bio-assembly 210 where a flowable suspension of cells is injected. In accordance with various embodiments, each of the one or more vascular components 235 can include different chambers or different compartment types in the bio-assembly 210 where different cell types are injected into different compartments.

In accordance with various embodiments, the bio-scaffold 230 optionally includes the void 238. In accordance with various embodiments, the one or more vascular components 235 is disposed in the void 238. In accordance with various embodiments, the bio-scaffold 230 includes one or more voids 238.

In accordance with various embodiments, the plate 220 includes the partition 250 that includes an internal volume 255. In accordance with various embodiments, the partition 250 is shaped to receive the bio-assembly 210 into the internal volume 255. In accordance with various embodiments, the plate 220 includes a plurality of partitions 250. In accordance with various embodiments, each of the plurality of partitions 250 includes an inner volume 255. In accordance with various embodiments, the plate 220 includes 1 to 1000 partitions, including 3, 4, 6, 12, 24, 48, 96, 192 or 384 partitions 250.

In accordance with various embodiments, the plate 220 includes identical material as the plate 120, and therefore will not be described in further detail.

In accordance with various embodiments, the plate 220 has similar dimensions as the plate 120, and therefore will not be described in further detail.

In accordance with various embodiments, the partition 250 has similar dimensions as the partition 150, and therefore will not be described in further detail.

In accordance with various embodiments, the loader 260 includes the loader inlet 262 and the loader outlet 264. In accordance with various embodiments, the loader inlet 262 and the loader outlet 264 are substantially orthogonal to a top surface of the plate 220. In accordance with various embodiments, the loader inlet 262 and the loader outlet 264 are substantially orthogonal to a top surface of the loader 260. In accordance with various embodiments, the loader inlet 262 and the loader outlet 264 are adjacent to each other and are disposed on the same side of the loader 260. In accordance with various embodiments, the loader inlet 262 and the loader outlet 264 are disposed on the different sides of the loader 260. In accordance with various embodiments, the loader inlet 262 and the loader outlet 264 are disposed on the opposite sides of the loader 260. In accordance with various embodiments, the loader inlet 262 and the loader outlet 264 are disposed on a top surface the loader 260. In accordance with various embodiments, the loader inlet 262 and the loader outlet 264 are disposed on a bottom surface the loader 260. In accordance with various embodiments, the loader inlet 262 and the loader outlet 264 have tapering or gradually tapering tips.

In accordance with various embodiments, the loader 260 includes identical material as the loader plate 120, and therefore will not be described in further detail.

In accordance with various embodiments, the loader 260 has similar dimensions as the loader plate 120, and therefore will not be described in further detail.

In accordance with various embodiments, the loader 260 can include more than one loader inlet 262 and more than one loader outlet 264. In accordance with various embodiments, the loader 260 can include up to 384 sets of loader inlets 262 and loader outlets 264. In accordance with various embodiments, the loader 260 can be configured to work with the plate 220 that includes 1 to 1000 partitions, including 3, 4, 6, 12, 24, 48, 96, 192 or 384 partitions 250.

In accordance with various embodiments, the loader inlet 262 and the loader outlet 264 are in fluid communication with the bio-scaffold 230. In accordance with various embodiments, the vascular inlet is in fluid communication with the loader inlet 262. In accordance with various embodiments, the vascular outlet is in fluid communication with the loader outlet 264. In accordance with various embodiments, each of the one or more vascular inlets is in fluid communication with an associated loader inlet 262 and each of the one or more vascular outlets is in fluid communication with an associated loader outlet 264.

In accordance with various embodiments, the bio-assembly 210 and loader fluid communication is mediated by a tapered constriction in the bio-assembly 210 and provides a fluidic seal at a normal operating fluid pressure during perfusion. In accordance with various embodiments, the fluidic seal is provided size differential between a larger size of the loader inlet 262/outlet 264 and smaller size of the vascular inlet/outlet of the bio-scaffold 230. In accordance with various embodiments, the mechanical fit between the female (vascular inlet/outlet of the bio-scaffold 230) and the male (the loader inlet 262/outlet 264) provides an “interference fit” between the male and female features. In accordance with various embodiments, an adapter may be used to provide a fluidic seal between the loader inlet 262/outlet 264 and the vascular inlet/outlet of the bio-scaffold 230.

In accordance with various embodiments, perfusing can occur under various perfusing mechanisms, such as for example, but not limited to under gravity flow, via a pump for positive pressure or via vacuum suctioning for negative pressure. In accordance with various embodiments, the normal operating fluid pressure is between about −100 kPa (negative pressure, such as, suction) and about 100 kPa (positive pressure, such as, pumped fluid, liquid or gas), between about −50 kPa and about 50 kPa, between about −15 kPa and about 15 kPa, between about −10 kPa and about 10 kPa, or between about −1 kPa and about 1 kPa.

In accordance with various embodiments, perfusing can occur at a fluid flow rate that does not shear the bio-scaffold 230. For example, perfusing can occur to mimic tidal ventilation, that may include positive pressure perfusion, with variations on flow like blood pumping (e.g., heart beat mimic), or continuous flow or within a flow regime without shearing the bio-scaffold 230. For example, perfusing can be performed with high-glucose media for about 3 hours, about 6 hours, about 9 hours, or about 12 hours followed by low glucose media for about 3 hours, about 6 hours, about 9 hours, or about 12 hours, mimicking when a person eats a meal.

In accordance with various embodiments, the bio-scaffold kit 200 optionally includes the adhesive 280. In accordance with various embodiments, the adhesive 280 is a bio-compatible adhesive positioned between the substrate 240 and the plate 220. In accordance with various embodiments, the adhesive 280 is configured to maintain a fluid-impermeable bond between the substrate 240 and the plate 220. In accordance with various embodiments, the adhesive 280 includes a degradable or bio-degradable material.

In accordance with various embodiments, the adhesive 280 includes identical material as the adhesive 180, and therefore will not be described in further detail.

In accordance with various embodiments, the bio-scaffold kit 200 optionally includes the ancillary component 290. In accordance with various embodiments, the ancillary component 290 can include any material that can flow within the one or more vascular component 235 or inside the void 238 of the bio-assembly 210. In accordance with various embodiments, the ancillary component 290 can include a fluid mixture having multiple fluid components. In accordance with various embodiments, the ancillary component 290 can include a fluid mixture that includes a liquid, foam, or secondary pre-matrix. In accordance with various embodiments, the ancillary component 290 can include a fluid mixture that can be injected into the bio-assembly 210.

In accordance with various embodiments, the void 238 of the bio-scaffold 230 of the bio-assembly 210 is a perfusable or injectable space with one or more inlets. In accordance with various embodiments, the void 238 of the bio-scaffold 230 of the bio-assembly 210 is a perfusable or injectable space with one or more outlets. In accordance with various embodiments, the ancillary component 290, such as the fluid mixture, can be configured to be combined with live cells, the combination being injectable into the void 238. In accordance with various embodiments, the void 238 includes physical anchors that the ancillary component 290, such as the fluid mixture is dispensed around.

In accordance with various embodiments, the ancillary component 290 includes perfusable media, such as for example, complete media, with oxygen carriers, with red blood cells, whole human blood, and/or de-fibrinated human blood that does not clot. In accordance with various embodiments, the ancillary component 290 can include a fluid or liquid, such as for example, but not limited to bile, blood, urine, lymph, and/or a gas within the one or more vascular component 235 or inside the void 238 of the bio-assembly 210. In accordance with various embodiments, the ancillary component 290 can include a material that can form parenchymal tissue, such as for example, but not limited to liver, kidney, pancreas, lung, heart, interstitial tissue, such as for example, but not limited to fibroblasts, mesenchymal stem cells (MSCs) and other matrix producing and support cells. In accordance with various embodiments, the ancillary component 290 can include a material that can form a “clean meat”, such as those that appear similar to a marbled structure of Kobe beef.

In accordance with various embodiments, the ancillary component 190 can include cells or cell types that can form one or more layers from the list consisting of an endothelial layer, an epithelial layer, a smooth muscle cell layer, a sequentially delivered smooth muscle cell layer and endothelial layer, a sequentially delivered smooth muscle cell layer and epithelial layer, a sequentially delivered smooth muscle cell layer, gel layer, endothelial or epithelial layer, a sequentially delivered pericyte layer and endothelial layer, or a sequentially delivered pericyte layer and epithelial layer.

In accordance with various embodiments, the ancillary component 290 includes 3D printable materials, such as for example, but not limited to interstitial cells, such as fibroblasts, hMSCs and endothelial cells within the bio-scaffold 230 of the bio-assembly 210.

In accordance with various embodiments, the ancillary component 290 can be included in the bio-scaffold kit 200 and cryopreserved at a temperature, for example, below 10° C., 0° C., −10° C., −25° C., −50° C., −75° C., −100° C., −150° C., −200° C., −250° C. or −270° C.

In accordance with various embodiments, a bio-scaffold system includes the bio-assembly 210 that can be tailored to adapt to tissue types by adding particular cells or ECM. In accordance with various embodiments, the bio-assembly 210 includes a blank bio-scaffold with architecture, whereby injected cells of the ancillary component 290 can take up residence, proliferate, migrate, invade the vasculature like metastasis. In accordance with various embodiments, the bio-scaffold system can be tailored under the control of exogenous factors similar to chemotherapeutics. In accordance with various embodiments, the bio-assembly 210 includes a blank bio-scaffold with architecture that can be injected with any type of cells to obtain an assay having a cell culture of that particular injected cell type. In accordance with various embodiments, the bio-assembly 210 includes a blank bio-scaffold with architecture that is injected with cancer cells to obtain a cancer invasion assay. In accordance with various embodiments, the bio-assembly 210 includes a blank bio-scaffold with architecture that is injected with liver cells to obtain a liver toxicology screening platform. In accordance with various embodiments, the bio-assembly 210 includes a blank bio-scaffold with architecture that is injected with cardiac cells to obtain a cardiac toxicology screening platform. In accordance with various embodiments, the bio-assembly 210 includes a blank bio-scaffold with architecture that is injected with kidney cells to obtain a kidney toxicology screening platform. In accordance with various embodiments, the bio-assembly 210 includes a blank bio-scaffold with architecture that is injected with brain cells to obtain a brain toxicology screening platform. In accordance with various embodiments, the bio-assembly 210 includes a blank bio-scaffold with architecture that is injected with gut cells to obtain a gut toxicology or gut permeability screening platform. In accordance with various embodiments, the bio-assembly 210 includes a blank bio-scaffold with architecture that is injected with lung cells to obtain a lung toxicology or gas transport screening platform. In accordance with various embodiments, the bio-assembly 210 offers identical tissue architecture suitable for high reproducibility and high throughput screening.

In accordance with various embodiments, the bio-assembly 210 is substantially transparent. In accordance with various embodiments, the bio-assembly 210 is transparent and suitable for imaging with visible light, fluorescence, and/or luminescence. In accordance with various embodiments, the bio-assembly 210 is transparent and suitable for imaging after histological, immunohistochemistry, or immunofluorescence staining following sectioning via a vibratome, microtome, or a cryostat machine. In accordance with various embodiments, the bio-assembly 210 includes regions that are non-cellularized regions that provide optical conduits for imaging.

In accordance with various embodiments, the loader 260 can include at least one loader inlet 262 and at least one loader outlet 262 in associated with each of the plurality of partitions 250. In accordance with various embodiments, the loader 260 further includes a fluid inlet channel and a fluid outlet channel. In accordance with various embodiments, the fluid inlet channel is in fluid communication with the loader inlet 262. In accordance with various embodiments, the fluid outlet channel is in fluid communication with the loader outlet 264. In accordance with various embodiments, the fluid inlet channel is in fluid communication with more than one loader inlet 262. In accordance with various embodiments, the fluid outlet channel is in fluid communication with more than one loader outlet 264.

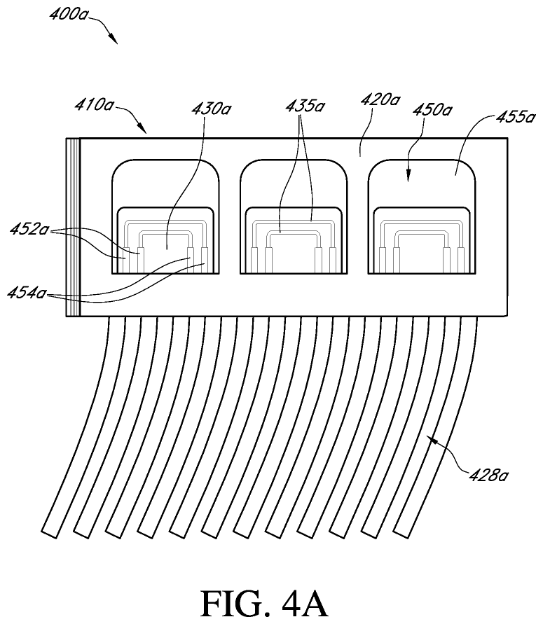

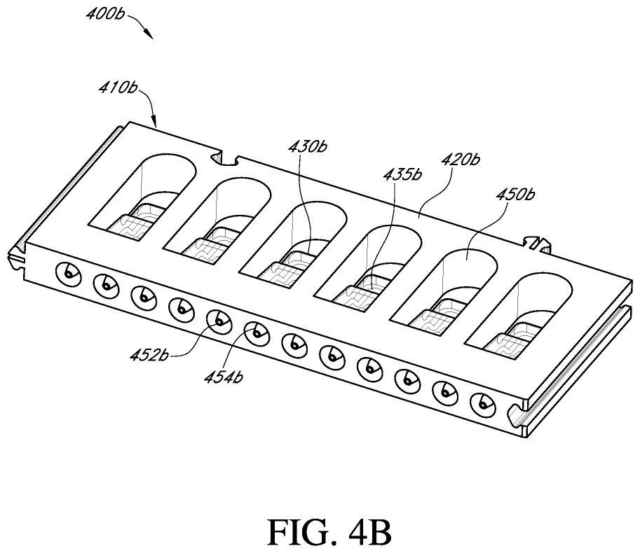

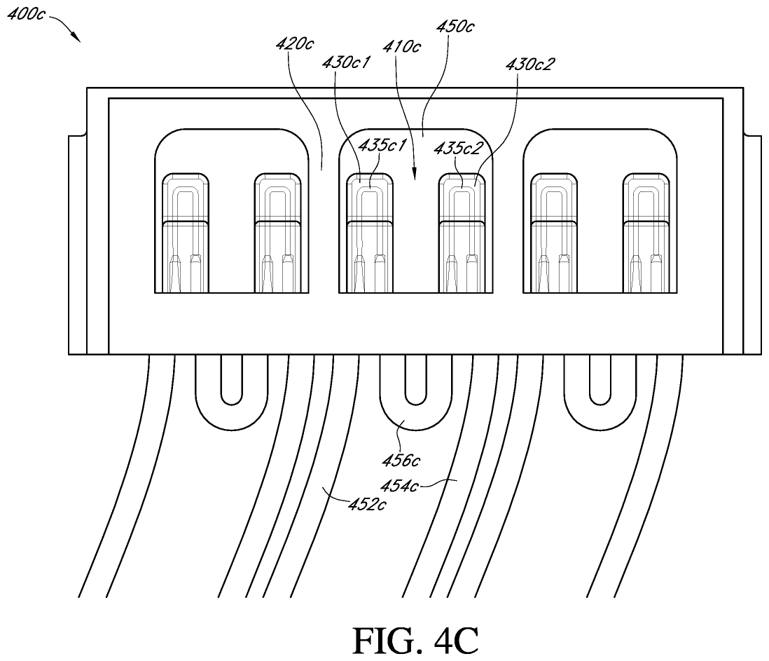

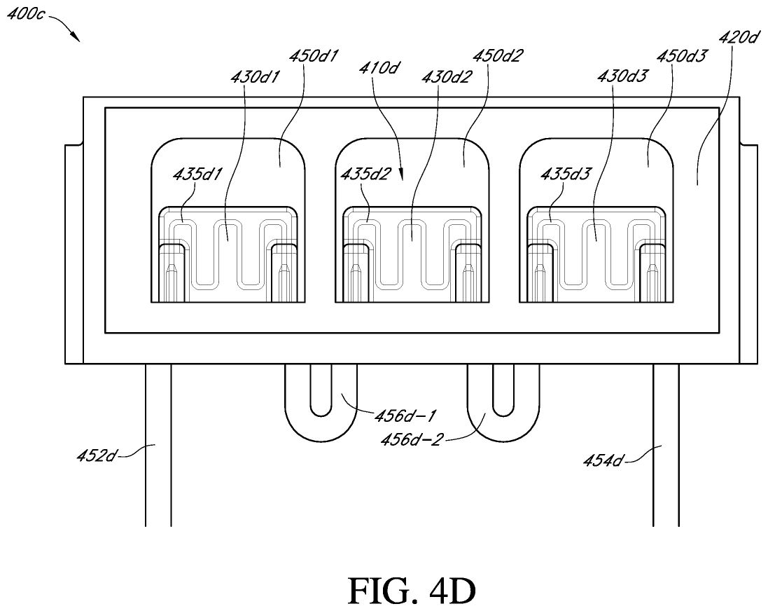

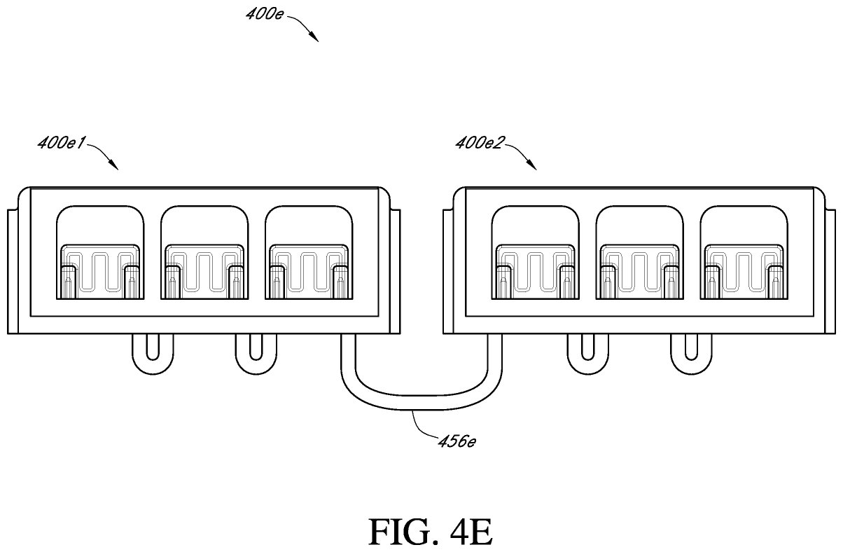

In accordance with various embodiments, the fluid outlet of one loader serves as the fluid inlet of one or more alternate loaders on the same device. In accordance with various embodiments, the fluid outlet of one loader servers as the fluid inlet of one or more alternate loaders on a different device. In accordance with various embodiments, the fluid outlet of one loader servers as the fluid inlet of one or more alternate loaders on the same device and/or on different devices. Various example connection schemes are described in further detail with respect to FIGS. 4A-4E. In accordance with various embodiments, a fluid outlet of one loader can serve as a fluid inlet of one or more alternative loaders on the same device, for example, such as interconnect 456d-1 of FIG. 4D. In accordance with various embodiments, a fluid outlet of one loader serves as a fluid inlet of one or more alternative loaders on a different device, for example, such as interconnect 456e of FIG. 4E. In accordance with various embodiments, one bio-scaffold can be connected to another bio-scaffold within the same loader plate by incorporating an inner channel in the loader plate to go from outlet to inlet instead of connecting tubing as shown with respect to FIG. 4C.

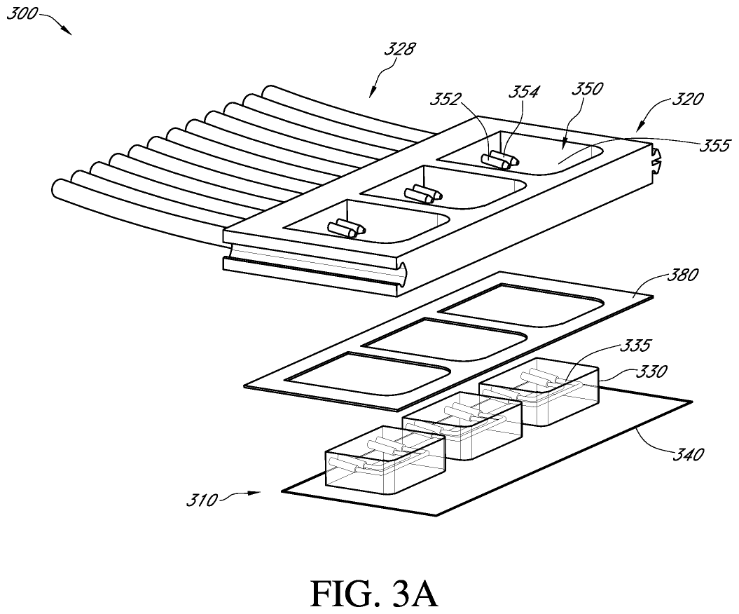

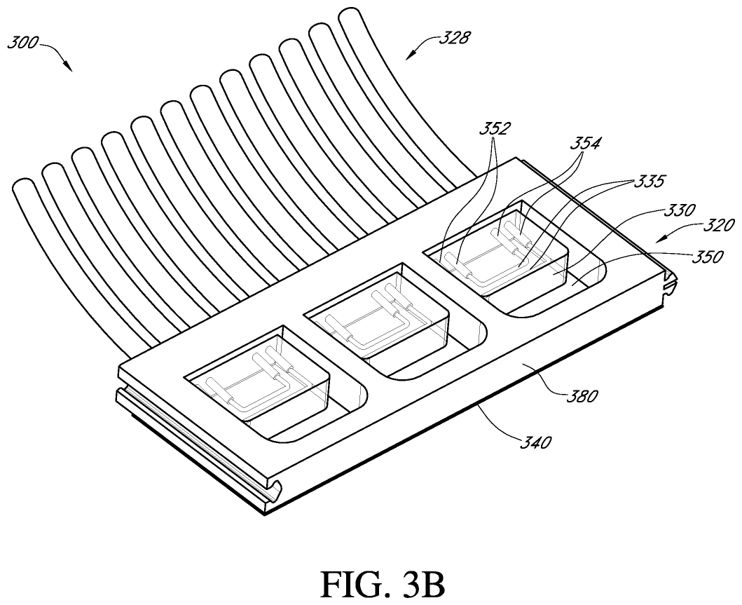

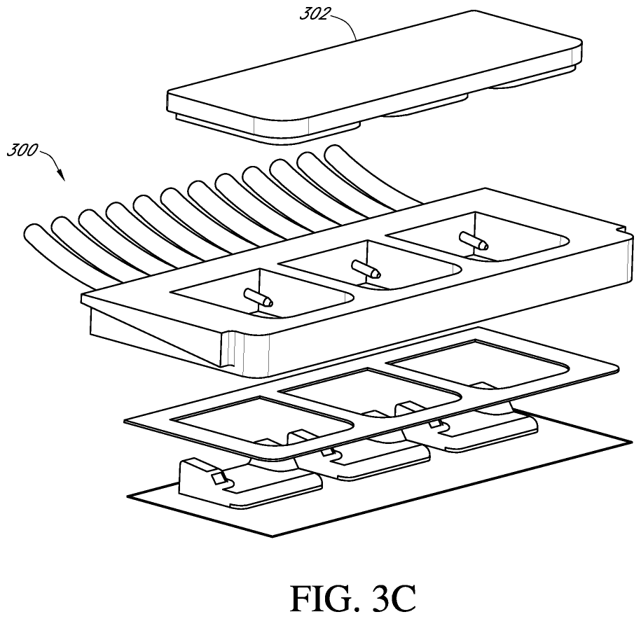

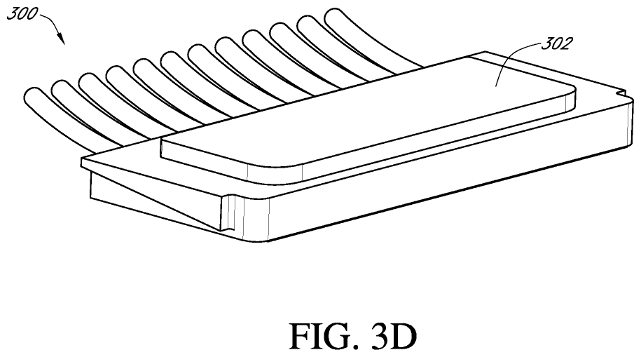

FIG. 3A is a schematic illustration of a bio-scaffold kit 300, according to various embodiments. FIG. 3B is another schematic illustration of the bio-scaffold kit 300 of FIG. 3A. The illustration of the bio-scaffold kit 300 of FIG. 3A is an exploded view of the assembled bio-scaffold kit 300 shown in FIG. 3B.

As shown in FIGS. 3A and 3B, the bio-scaffold kit 300 includes a bio-assembly 310, a loader plate 320, and an adhesive 380. In accordance with various embodiments, the bio-assembly 310 includes a bio-scaffold 330. In accordance with various embodiments, the bio-assembly 310 includes a substrate 340. In accordance with various embodiments, the bio-scaffold 330 includes a vascular component 335. In accordance with various embodiments, the loader plate 320 includes a partition 350. In accordance with various embodiments, the partition 350 can include a partition inlet 352 and a partition outlet 354.

In accordance with various embodiments, the partition 350 can have openings configured for inlet or outlet. In accordance with various embodiments, the bio-scaffold 330 can be chemically bound to partition openings (e.g., without the partition inlet 352 or outlet 354). In accordance with various embodiments, the chemical bond can result in a seal between the bio-scaffold 330 and the partition 350 for enabling a flow of a fluid mixture across the two components.

In accordance with various embodiments, the bio-scaffold kit 300 can also include an ancillary component. In accordance with various embodiments, the bio-scaffold kit 300 includes identical material as the bio-scaffold kit 100, and therefore will not be described in further detail, unless described otherwise. As shown in FIGS. 3A and 3B, tubings 328 are connected to the partition inlet 352 and the partition outlet 354 and configured to perfuse the ancillary component.

In accordance with various embodiments, the bio-assembly 310 includes the bio-scaffold 330 affixed or otherwise disposed on the substrate 340. In accordance with various embodiments, the bio-scaffold 330 is affixed or otherwise disposed on the substrate 340 via any suitable bonding techniques, including for example, but not limited to, covalently bonding the bio-scaffold 330 to a top surface of the substrate 340, which can be either functionalized with silane or any other means to promote adhesion between the bio-scaffold 330 and the substrate 340. In accordance with various embodiments, adhesive can include tape, liquid adhesive/glue, or UV curable materials, or any other suitable materials. In accordance with various embodiments, a substrate is a glass slide that is intimately in contact with the partition. In accordance with various embodiments, the bio-scaffold 330 is a hydrogel that can be disposed on the substrate 340 without covalent bonding. In accordance with various embodiments, the bio-scaffold 330 can be disposed on the substrate 340.

In accordance with various embodiments, the substrate 340 can be used as a substrate in cell culturing environments. In accordance with various embodiments, the substrate 340 can be transparent glass or plastics, or any other suitable material, such as for example, but not limited to polycarbonate, polysulfone, polymethyl methacrylate, polystyrene, cyclic olefin copolymer, polyethylene, polypropylene, glass, quartz, mica, infrared-transparent salts, such as calcium bromide, potassium bromide, or any of these materials combined with a thin film of any other material, or with a thin metallic film to enable surface plasmon based measurements.

In accordance with various embodiments, the bio-scaffold 330 includes identical material as the bio-scaffold 130, and therefore will not be described in further detail.

In accordance with various embodiments, the bio-scaffold 330 includes one or more vascular components 335. In FIG. 3A, the bio-scaffold 330 is shown to include two vascular components 335. In accordance with various embodiments, the vascular components 335 includes two vascular inlets and two vascular outlets. In accordance with various embodiments, the vascular components 335 can include one or more channels that may branch out as a tree-like structure within the bio-scaffold 330. In accordance with various embodiments, the one or more channels of the vascular components 335 may include branches that can form, for example, as a torus knot, wherein the channels re-converge at another point within the bio-scaffold 330. In accordance with various embodiments, the vascular components 335 can include branched structures that can extend from various portions of the bio-scaffold 330 and terminate at other portions within the bio-scaffold 330. In accordance with various embodiments, the one or more vascular components 335 can have a multiscale vasculature having branches and taperings similar to that of organs in human body.

In accordance with various embodiments, the components 335 have one or more channels of any shape in cross-section or aspect ratios that have a cross-section dimension or width (e.g., the cross-section dimension is a diameter if circular) ranging from about 10 μm to about 1 mm, 100 μm to about 500 μm, or about 800 microns or less, about 500 microns or less, about 400 microns or less, about 300 microns or less, or 200 microns or less. In accordance with various embodiments, the vascular components 335 are perfusable. In accordance with various embodiments, the one or more channels of the vascular components 335 are expandable in response to increases in pressure, mechanical, electrical, and/or chemical stimuli within the one or more vascular components 335. In accordance with various embodiments, the one or more channels of the one or more vascular components 335 are contractable in response to pressure, mechanical, electrical, and/or chemical stimuli within the one or more vascular components 335.

In accordance with various embodiments, the vascular components 335 can include a narrowing inlet and a narrowing outlet. In accordance with various embodiments, the vascular inlet and vascular outlet for a first vascular component of the vascular components 335 is positioned substantially orthogonal to the vascular inlet and vascular outlet of a second vascular component of the vascular components 335.

In accordance with various embodiments, each of the vascular components 335 can include a chamber or compartment in the bio-assembly 310 where a flowable suspension of cells is injected. In accordance with various embodiments, each of the vascular components 335 can include different chambers or different compartment types in the bio-assembly 310 where different cell types are injected into different compartments. In accordance with various embodiments, the bio-scaffold 330 optionally includes a void.

In accordance with various embodiments, the loader plate 320 includes the partition 350 which includes the partition inlet 352 and the partition outlet 354. In accordance with various embodiments, the partition inlet 352 and the partition outlet 354 are substantially parallel to a top surface of the loader plate 320. In accordance with various embodiments, the partition inlet 352 and the partition outlet 354 are adjacent to each other and are disposed on the same side of the loader plate 320. In accordance with various embodiments, the partition inlet 352 and the partition outlet 354 are disposed on the different sides of the loader plate 320. In accordance with various embodiments, the partition inlet 352 and the partition outlet 354 are disposed on the opposite sides of the loader plate 320. In accordance with various embodiments, the partition inlet 352 and the partition outlet 354 have tapering or gradually tapering tips.

In accordance with various embodiments, the loader plate 320 includes a plurality of partitions 350. In accordance with various embodiments, each of the plurality of partitions 350 includes a partition inlet 352 and the partition outlet 354. In accordance with various embodiments, the loader plate 320 includes 1 to 1000 partitions, including 3, 4, 6, 12, 24, 48, 96, 192 or 384 partitions 350. In accordance with various embodiments, each of the plurality of partitions 350 includes at least one set, and at most 20 sets, of partition inlets 352 and partition outlets 354.

In accordance with various embodiments, the loader plate 320 includes the partition 350 that includes an internal volume 355. In accordance with various embodiments, each of the plurality of partitions 350 includes an inner volume 355. In accordance with various embodiments, each of the plurality of partitions 350 is shaped to receive the bio-assembly 310 into the internal volume 355.

In accordance with various embodiments, the partition inlet 352 and the partition outlet 354 are in fluid communication with the bio-scaffold 330. In accordance with various embodiments, the vascular inlet is in fluid communication with the partition inlet 352. In accordance with various embodiments, the vascular outlet is in fluid communication with the partition outlet 354. In accordance with various embodiments, each of the one or more vascular inlets is in fluid communication with an associated partition inlet and each of the one or more vascular outlets is in fluid communication with an associated partition outlet.

In accordance with various embodiments, the bio-assembly 310 and partition fluid communication is mediated by a tapered constriction in the bio-assembly 310 and provides a fluidic seal at a normal operating fluid pressure during perfusion. In accordance with various embodiments, the fluidic seal is provided size differential between a larger size of the inlet 352/outlet 354 of the partition 350 and smaller size of the inlet/outlet of the bio-scaffold 330. In accordance with various embodiments, the mechanical fit between the female (inlet/outlet of the bio-scaffold 330) and the male (the inlet 352/outlet 354 of the partition 350) provides an interference fit between the male and female features. In accordance with various embodiments, an adapter may be used to provide a fluidic seal between the inlet 352/outlet 354 of the partition 350 and the inlet/outlet of the bio-scaffold 330.

In accordance with various embodiments, perfusing can occur under various perfusing mechanisms, such as for example, but not limited to under gravity flow, via a pump for positive pressure or via vacuum suctioning for negative pressure. In accordance with various embodiments, the normal operating fluid pressure is between about −100 kPa (negative pressure, such as, suction) and about 100 kPa (positive pressure, such as, pumped fluid, liquid or gas), between about −50 kPa and about 50 kPa, between about −15 kPa and about 15 kPa, between about −10 kPa and about 10 kPa, or between about −1 kPa and about 1 kPa.

In accordance with various embodiments, the bio-scaffold kit 300 includes the adhesive 380. In accordance with various embodiments, the adhesive 380 is a bio-compatible adhesive positioned between the substrate 340 and the loader plate 320. In accordance with various embodiments, the adhesive 380 is configured to maintain a fluid-impermeable bond between the substrate 340 and the loader plate 320. In accordance with various embodiments, the adhesive 380 includes a degradable or bio-degradable material.

In accordance with various embodiments, the adhesive 380 includes identical material as the adhesive 180, and therefore will not be described in further detail.

In accordance with various embodiments, the bio-scaffold kit 300 optionally includes the ancillary component. In accordance with various embodiments, the ancillary component can include any material that can flow within the vascular components 335. In accordance with various embodiments, the ancillary component can include a fluid mixture having multiple fluid components. In accordance with various embodiments, the ancillary component can include a fluid mixture that includes a liquid, foam, or secondary pre-matrix. In accordance with various embodiments, the ancillary component can include a fluid mixture that can be injected into the bio-assembly 310.

In accordance with various embodiments, the vascular components 335 of the bio-scaffold 330 of the bio-assembly 310 is a perfusable or injectable space with one or more inlets. In accordance with various embodiments, the vascular components 335 is a perfusable or injectable space with one or more outlets. In accordance with various embodiments, the ancillary component, such as the fluid mixture, can be combined with live cells before injection into the vascular components 335. In accordance with various embodiments, the vascular components 335 includes physical anchors that the ancillary component, such as the fluid mixture is dispensed around.

In accordance with various embodiments, the ancillary component is identical to the ancillary component 190 and therefore will not be discussed in further detail.

In accordance with various embodiments, a bio-scaffold system includes the bio-assembly 310 that can be tailored to adapt to tissue types by adding particular cells or ECM. In accordance with various embodiments, the bio-assembly 310 includes a blank bio-scaffold with architecture, whereby injected cells of the ancillary component can take up residence, proliferate, migrate, invade the vasculature like metastasis. In accordance with various embodiments, the bio-scaffold system can be tailored under the control of exogenous factors similar to chemotherapeutics. In accordance with various embodiments, the bio-assembly 310 includes a blank bio-scaffold with architecture that can be injected with any type of cells to obtain an assay having a cell culture of that particular injected cell type. In accordance with various embodiments, the bio-assembly 310 includes a blank bio-scaffold with architecture that is injected with cancer cells to obtain a cancer invasion assay. In accordance with various embodiments, the bio-assembly 310 includes a blank bio-scaffold with architecture that is injected with liver cells to obtain a liver toxicology screening platform. In accordance with various embodiments, the bio-assembly 310 offers identical tissue architecture suitable for high reproducibility and high throughput screening.

In accordance with various embodiments, the bio-assembly 310 is substantially transparent. In accordance with various embodiments, the bio-assembly 310 is transparent and suitable for imaging with visible light, fluorescence, and/or luminescence. In accordance with various embodiments, the bio-assembly 310 includes regions that are non-cellularized regions that provide optical conduits for imaging.

FIGS. 3C and 3D are schematic illustrations of the bio-assembly kit 300 with a lid 302, in accordance with various embodiments. In accordance with various embodiments, the lid 302 is configured to removably cover or close the partition 350 so that the internal volume 355 is enclosed, as shown in FIGS. 3C and 3D. In accordance with various embodiments, the lid 302 can be tight fitting for sealing or loose fitting so that addition of fluid/cells/biomaterials can be performed easily. In accordance with various embodiments, the covered partition 350 of the bio-assembly kit 300 can maintain the desired operation parameters within the internal volume 355, e.g., to prevent the fluid mixture in the bio-assembly 310 from evaporating or drying out. The use of a lid, such as the lid 302, or similar type, mechanism, or configuration can be applied and used in any of the embodiments shown and described with respect to FIGS. 4-7 as discussed herein.

FIG. 4A is a schematic illustration of an example bio-scaffold kit 400a, according to various embodiments. As illustrated in FIG. 4A, the bio-scaffold kit 400a, shown in top-view, includes a bio-assembly 410a that includes a bio-scaffold 430a, and a loader plate 420a. In accordance with various embodiments, the bio-scaffold 430a includes two vascular components 435a. In accordance with various embodiments, the loader plate 420a includes partitions 450a, each having an inner volume 455a. As shown in FIG. 4A, each of the partitions 450a includes partition inlets 452a and partition outlets 454a.

In accordance with various embodiments, the bio-scaffold kit 400a can also include an ancillary component. In accordance with various embodiments, tubings 328 are connected to the partition inlets 452a and partition outlets 454a, and are configured to perfuse the ancillary component. In accordance with various embodiments, the bio-scaffold kit 400a is the same or substantially similar to the bio-scaffold kit 300, and therefore will not be described in further detail.

FIG. 4B is a schematic illustration of another example bio-scaffold kit 400b, according to various embodiments. In accordance with various embodiments, the bio-scaffold kit 400b is substantially similar to the bio-scaffold kit 400a shown in FIG. 4A. As illustrated in FIG. 4B, the bio-scaffold kit 400b, shown in perspective view, includes a bio-assembly 410b that includes a bio-scaffold 430b, and a loader plate 420b. In accordance with various embodiments, the bio-scaffold 430b includes a single vascular component 435b. In accordance with various embodiments, each of the partitions 450b includes a single partition inlet 452b and a single partition outlet 454b. In accordance with various embodiments, the bio-scaffold kit 400b shown and described with respect to FIG. 4B is similar to the bio-scaffold kit 400a shown and described with respect to FIG. 4A, except that each of the partitions 450b includes a single partition inlet 452b and a single partition outlet 454b whereas each of the partitions 450b includes two partition inlets 452b and two partition outlets 454b.

FIG. 4C is a schematic illustration of another example bio-scaffold kit 400c, according to various embodiments. As illustrated in FIG. 4C, the bio-scaffold kit 400c, shown in top-view, includes a bio-assembly 410c that includes two bio-scaffolds 430c1 and 430c2 disposed within a single partition 450c (center partition in FIG. 4C) of a loader plate 420c. In accordance with various embodiments, the bio-scaffolds 430c1 and 430c2 each includes a single vascular component, 435c1 and 435c2, respectively. In accordance with various embodiments, each of the partitions 450c includes a partition inlet 452c and a partition outlet 454c. As shown in FIG. 4C, the partition inlet 452c is connected to an inlet of the single vascular component 435c1, an outlet of the single vascular component 435c1 is then connected via an interconnect 456c to an inlet of the single vascular component 435c2, and an outlet of the single vascular component 435c2 is connected to the partition outlet 454c. In accordance with various embodiments, FIG. 4C illustrates a daisy-chain structure that is formed between adjacent bio-scaffolds 430c1 and 430c2 within a single partition 450c. A daisy-chain structure is defined as a connection scheme in which multiple devices or components are connected in sequence. For example, daisy-chaining different devices or different loaders within the same device, is common across all the implementations as shown and described herein. In accordance with various embodiments, the daisy-chain structure or configuration illustrated in FIG. 4C allows connecting two bio-scaffolds 430c1 and 430c2 within a single bio-assembly 410c to be in fluid communication, and thus the structure is configurable to flow the same fluid or fluid mixture at a single rate. In accordance with various embodiments, adjacent partitions 450c to the center partition can be configured to flow a different or the same fluid or fluid mixture at a different (and independently) flow rate or the same flow rate. In accordance with various embodiments, flowing at a different flow rate and independently encompasses using a separate controllable pump, pumping mechanism, or suctioning mechanism.

FIG. 4D is a schematic illustration of another example bio-scaffold kit 400d, according to various embodiments. As illustrated in FIG. 4D, the bio-scaffold kit 400d, shown in top-view, includes a bio-assembly 410d and a loader plate 420d. In accordance with various embodiments, the loader plate 420d includes three partitions 450d1, 450d2, and 450d3. In accordance with various embodiments, the bio-assembly 410d includes three bio-scaffolds 430d1, 430d2, and 430d3 disposed within each of the respective partitions 450d1, 450d2, and 450d3, as shown in FIG. 4D. Although shown to include only three sets of partitions and bio-scaffolds , any number of partitions and bio-scaffolds can be used in accordance with various embodiments.

In accordance with various embodiments, the bio-scaffolds 430d1, 430d2, and 430d3 each respectively includes a vascular component 435d1, 435d2, and 435d3, as shown in FIG. 4D. Although shown to include a single vascular component in each of the bio-scaffolds, any number of vascular components can be included in each of the bio-scaffolds, in accordance with various embodiments. In accordance with various embodiments, the loader plate 420d includes a partition inlet 452d and a partition outlet 454d. As shown in FIG. 4C, the partition inlet 452d is connected to an inlet of the vascular component 435d1, an outlet of the vascular component 435d1 is connected via an interconnect 456d-1 to an inlet of the vascular component 435d2, an outlet of the vascular component 435d2 is connected via an interconnect 456d-2 to an inlet of the vascular component 435d3, and an outlet of the vascular component 435d3 is connected to the partition outlet 454d. In accordance with various embodiments, FIG. 4D illustrates a daisy-chain structure that is formed between bio-scaffolds 430d1, 430d2, and 430d3 that reside within their respective partitions 450d1, 450d2, and 450d3. In accordance with various embodiments, the daisy-chain structure or configuration illustrated in FIG. 4D allows connecting three bio-scaffolds 430d1, 430d2, and 430d3 disposed within three separate partitions 450d1, 450d2, and 450d3 to be in fluid communication. In accordance with various embodiments, the structure of FIG. 4D is configurable to flow the same fluid or fluid mixture at a single rate via the inlet 452d and the outlet 454d using a pump, pumping mechanism, or suctioning mechanism.

FIG. 4E is a schematic illustration of another example bio-scaffold kit 400e, according to various embodiments. As illustrated in FIG. 4E, the bio-scaffold kit 400e, shown in top-view, includes two bio-scaffold kit 400e1 and 400e2 that are connected via an interconnect 456e. In accordance with various embodiments, FIG. 4E illustrates a daisy-chain structure that is formed between adjacent bio-scaffold kits. Although shown to include only two bio-scaffold kits, any number of bio-scaffold kits can be interconnected or daisy-chained. In accordance with various embodiments, the bio-scaffold kits 400e1 or 400e2 can be replaced with any of the bio-scaffold kits 100, 200, 300, 400a, 400b, 400c, or 400d, and/or can be connected or interconnected in any possible configurations to be in fluid communication, for example, either to flow the same fluid or fluid mixture or different fluid or fluid mixture, at either a single rate or different rates that can be independently controlled, via any pump, pumping mechanism, or suctioning mechanism. In other words, the disclosed configurations, embodiments, and various implementation types, or configuration schemes described herein are simply to illustrate the various possible combinations of embodiments and examples, and thus these are by no means limiting only to the illustrations and examples. In accordance with various embodiments, any possible combination and permutations of the various disclosed structures can be employed and applicable, and thus only limited by the imagination of a skilled artisan.

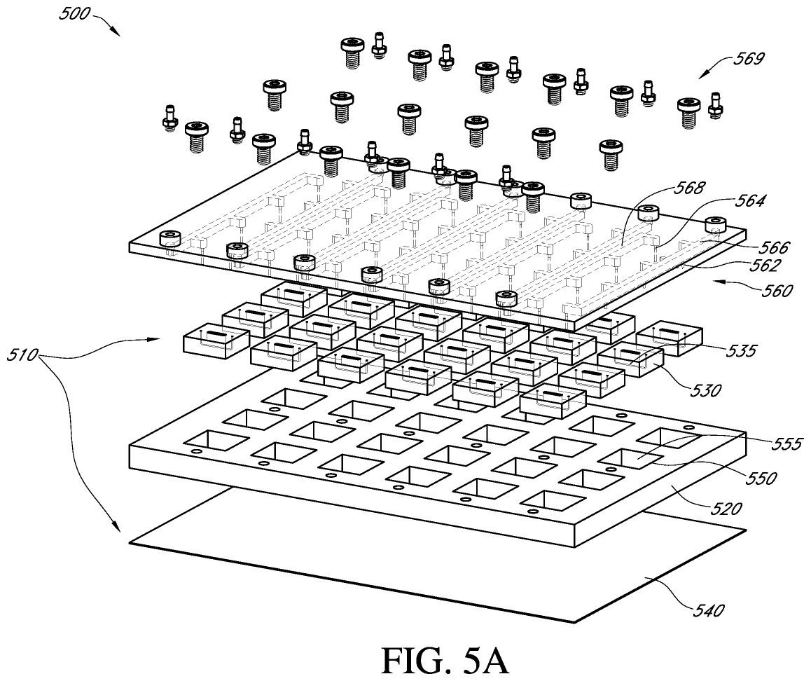

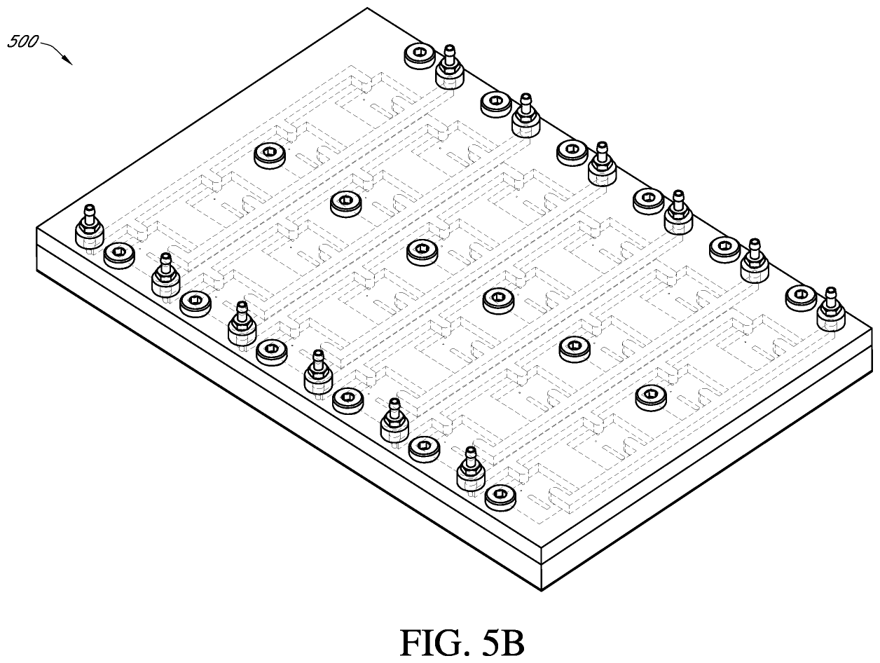

FIG. 5A is a schematic illustration of a bio-scaffold kit 500, according to various embodiments. FIG. 5B is another schematic illustration of the bio-scaffold kit 500 of FIG. 5A. The illustration of the bio-scaffold kit 500 of FIG. 5A is an exploded view of the assembled bio-scaffold kit 500 shown in FIG. 5B.

As shown in FIGS. 5A and 5B, the bio-scaffold kit 500 includes a bio-assembly 510, a plate 520, a loader 560 and can optionally include adhesive and/or ancillary component. In accordance with various embodiments, the bio-assembly 510 includes a bio-scaffold 530. As shown in FIG. 5A, the bio-assembly 510 includes a substrate 540. In accordance with various embodiments, the bio-scaffold 530 includes a vascular component 535. In accordance with various embodiments, the bio-scaffold 530 optionally includes a void. In accordance with various embodiments, the plate 520 includes a partition 550. In accordance with various embodiments, the loader 560 includes a loader inlet 562 and a loader outlet 564. In accordance with various embodiments, the loader 560 includes a fluid inlet channel 566 and a fluid outlet channel 568. As shown in FIG. 5A, the bio-scaffold kit 500 may also include various other components 569 that help with securing fluid inlets and outlets.

In accordance with various embodiments, the bio-scaffold 530 includes identical material as the bio-scaffold 130, and therefore will not be described in further detail. In accordance with various embodiments, the bio-scaffold 530 includes one or more vascular components 535, although only one vascular component 535 is illustrated in FIG. 5A. In accordance with various embodiments, the bio-scaffold 530 along with the one or more vascular components 535 can be 3D printed or molded. In accordance with various embodiments, each of the one or more vascular component 535 includes a vascular inlet and a vascular outlet. In accordance with various embodiments, the one or more vascular components 535 includes one or more channels that may branch out as a tree-like structure within the bio-scaffold 530. In accordance with various embodiments, the one or more channels of the one or more vascular components 535 may include branches that can form, for example, as a torus knot, wherein the channels re-converge at another point within the bio-scaffold 530. In accordance with various embodiments, the one or more vascular components 535 can include branched structures that can extend from various portions of the bio-scaffold 530 and terminate at other portions within the bio-scaffold 530. In accordance with various embodiments, the one or more vascular components 535 can have a multiscale vasculature having branches and taperings similar to that of organs in human body.

In accordance with various embodiments, the one or more vascular components 535 have one or more channels of any shape in cross-section or aspect ratios that have a cross-section dimension or width (e.g., the cross-section dimension is a diameter if circular) ranging from about 10 μm to about 1 mm, 100 μm to about 500 μm, or about 800 microns or less, about 500 microns or less, about 400 microns or less, about 300 microns or less, or 200 microns or less. In accordance with various embodiments, the one or more vascular components 535 are perfusable. In accordance with various embodiments, the one or more channels of the one or more vascular components 535 are expandable in response to increases in pressure, mechanical, electrical, and/or chemical stimuli within the one or more vascular components 535. In accordance with various embodiments, the one or more channels of the one or more vascular components 535 are contractable in response to pressure, mechanical, electrical, and/or chemical stimuli within the one or more vascular components 535.

In accordance with various embodiments, the one or more vascular components 535 can include a narrowing inlet and a narrowing outlet. In accordance with various embodiments, the one or more vascular components 535 can include one or more vascular inlets. In accordance with various embodiments, the one or more vascular components 535 can include one or more vascular outlets. In accordance with various embodiments, each of the one or more vascular components 535 can include a vascular inlet and a vascular outlet. In accordance with various embodiments, the vascular inlet and vascular outlet for a first vascular component of the one or more vascular components 535 is positioned orthogonal or substantially orthogonal, parallel or substantially parallel, or at an angle between 0 degree and 90 degrees, with respect to the vascular inlet and vascular outlet of a second vascular component of the one or more vascular components 535.

In accordance with various embodiments, each of the one or more vascular components 535 can include a chamber or compartment in the bio-assembly 510 where a flowable suspension of cells is injected. In accordance with various embodiments, each of the one or more vascular components 535 can include different chambers or different compartment types in the bio-assembly 510 where different cell types are injected into different compartments.

In accordance with various embodiments, the bio-scaffold 530 optionally includes a void. In accordance with various embodiments, the one or more vascular components 535 is disposed in the void.

In accordance with various embodiments, the plate 520 includes the partition 550 that includes an internal volume 555. In accordance with various embodiments, the partition 550 is shaped to receive the bio-assembly 510 into the internal volume 555. As shown in FIG. 5A, the plate 520 includes a plurality of partitions 550 arranged in an array. In accordance with various embodiments, each of the plurality of partitions 550 includes an inner volume 555. In accordance with various embodiments, the plate 520 includes 1 to 1000 partitions, including 3, 4, 6, 12, 24, 48, 96, 192 or 384 partitions 550.

In accordance with various embodiments, the loader 560 includes the loader inlet 562 and the loader outlet 564. In accordance with various embodiments, the loader inlet 562 and the loader outlet 564 are substantially orthogonal to a top surface of the plate 520. In accordance with various embodiments, the loader inlet 562 and the loader outlet 564 are substantially orthogonal to a top surface of the loader 560. In accordance with various embodiments, the loader inlet 562 and the loader outlet 564 are adjacent to each other and are disposed on the same side of the loader 560. In accordance with various embodiments, the loader inlet 562 and the loader outlet 564 are disposed on the different sides of the loader 560. In accordance with various embodiments, the loader inlet 562 and the loader outlet 564 are disposed on the opposite sides of the loader 560. In accordance with various embodiments, the loader inlet 562 and the loader outlet 564 are disposed on a top surface the loader 560. In accordance with various embodiments, the loader inlet 562 and the loader outlet 564 are disposed on a bottom surface the loader 560. In accordance with various embodiments, the loader inlet 562 and the loader outlet 564 have tapering or gradually tapering tips.

In accordance with various embodiments, the loader 560 can include more than one loader inlet 562 and more than one loader outlet 564. In accordance with various embodiments, the loader 560 can include up to 384 sets of loader inlets 562 and loader outlets 564. In accordance with various embodiments, the loader 560 can be configured to work with the plate 520 that includes 1 to 1000 partitions, including 3, 4, 6, 12, 24, 48, 96, 192 or 384 partitions 550.

In accordance with various embodiments, the loader inlet 562 and the loader outlet 564 are in fluid communication with the bio-scaffold 530. In accordance with various embodiments, the vascular inlet is in fluid communication with the loader inlet 562. In accordance with various embodiments, the vascular outlet is in fluid communication with the loader outlet 564. In accordance with various embodiments, each of the one or more vascular inlets is in fluid communication with an associated loader inlet 562 and each of the one or more vascular outlets is in fluid communication with an associated loader outlet 564.

In accordance with various embodiments, the bio-assembly 510 and loader fluid communication is mediated by a tapered constriction in the bio-assembly 510 and provides a fluidic seal at a normal operating fluid pressure during perfusion. In accordance with various embodiments, perfusing can occur under various perfusing mechanisms, such as for example, but not limited to under gravity flow, via a pump for positive pressure or via vacuum suctioning for negative pressure. In accordance with various embodiments, the normal operating fluid pressure is between about −100 kPa (negative pressure, such as, suction) and about 100 kPa (positive pressure, such as, pumped fluid, liquid or gas), between about −50 kPa and about 50 kPa, between about −15 kPa and about 15 kPa, between about −10 kPa and about 10 kPa, or between about −1 kPa and about 1 kPa.

In accordance with various embodiments, the bio-scaffold kit 500 optionally includes the ancillary component. In accordance with various embodiments, the bio-scaffold kit 500 is the same or substantially similar to the bio-scaffold kit 200 as described with respect to FIG. 2, and therefore will not be described in further detail.

In accordance with various embodiments, the loader 560 can include at least one loader inlet 562 and at least one loader outlet 562 in associated with each of the plurality of partitions 550. In accordance with various embodiments, the loader 260 further includes the fluid inlet channel 566 and the fluid outlet channel 568. In accordance with various embodiments, the loader 260 further includes more than one fluid inlet channel 566 and more than one fluid outlet channel 568. As shown in FIG. 5A, each of the fluid inlet channels 566 and the fluid outlet channels 568 are in fluid communication with more than one loader inlet 562 and outlet 564. In accordance with various embodiments, the fluid inlet channel 566 is in fluid communication with the loader inlet 562. In accordance with various embodiments, the fluid outlet channel 568 is in fluid communication with the loader outlet 564. In accordance with various embodiments, the fluid inlet channel 566 is in fluid communication with more than one loader inlet 562. In accordance with various embodiments, the fluid outlet channel 568 is in fluid communication with more than one loader outlet 564. In accordance with various embodiments, the fluid outlet of one loader serves as the fluid inlet of one or more alternate loaders on the same device. In accordance with various embodiments, the fluid outlet of one loader servers as the fluid inlet of one or more alternate loaders on a different device. In accordance with various embodiments, the fluid outlet of one loader servers as the fluid inlet of one or more alternate loaders on the same device and/or on different devices. In accordance with various embodiments, a single inlet of the bio-scaffold kit 500 is in fluid communication with one or more fluid inlet channels, such as the fluid inlet channel 566, in series and/or parallel. In accordance with various embodiments, a single inlet of the bio-scaffold kit 500 is in fluid communication with one or more bio-scaffolds 530 in series and/or parallel via one or more fluid inlet channels, such as the fluid inlet channel 566. In accordance with various embodiments, a single outlet of the bio-scaffold kit 500 is in fluid communication with one or more fluid outlet channels, such as the fluid outlet channel 568, in series and/or parallel. In accordance with various embodiments, a single outlet of the bio-scaffold kit 500 is in fluid communication with one or more bio-scaffolds 530 in series and/or parallel via one or more fluid outlet channels, such as the fluid outlet channel 568.

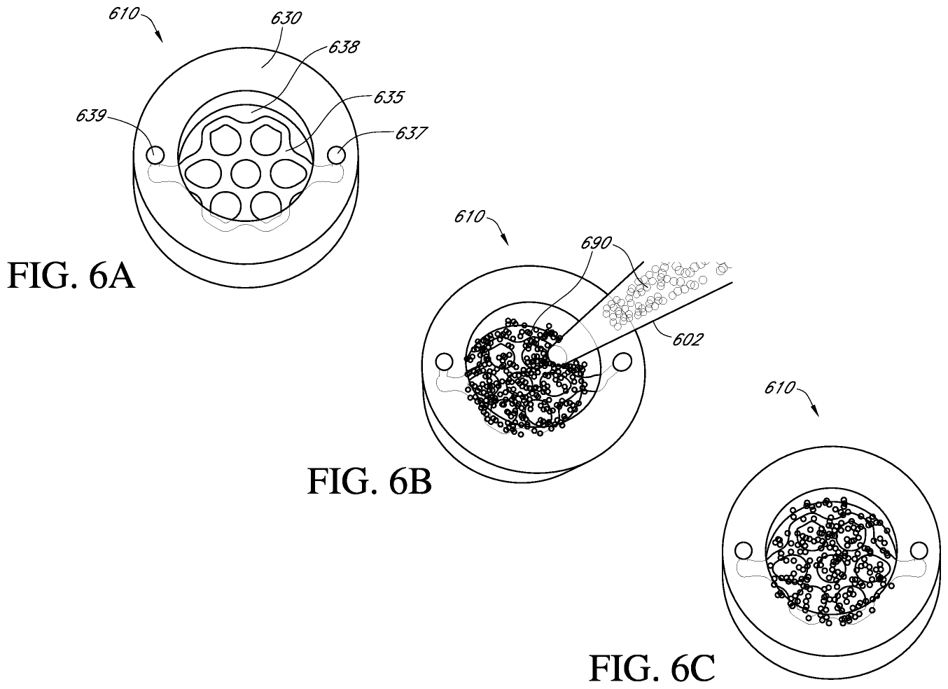

FIGS. 6A, 6B, 6C illustrate various stages of cell loading in an example bio-assembly 610, according to various embodiments. As shown in FIG. 6A, the bio-assembly 610 includes a bio-scaffold 630 that includes a vascular component 635 and a void 638. In accordance with various embodiments, the vascular component 635 includes a vascular inlet 637 and a vascular outlet 639. In accordance with various embodiments, the bio-assembly 610, the bio-scaffold 630, the vascular component 635, the vascular inlet 637, the vascular outlet 639, and the void 638 are similar or identical to the bio-assemblies 110 and/or 210, the bio-scaffolds 130 and/or 230, the vascular components 135 and/or 235, the vascular inlets, the vascular outlets, and the voids as described with respect to FIGS. 1 and 2, and therefore will not be described in further detail. As illustrated in FIG. 6B, various cells or cell types 690 can be disposed in the void 638 of the bio-assembly 610 via the use of a pipet 602. FIG. 6C illustrates the bio-assembly 610 having the various cells or cell types 690 in the void 638, which can be used for cell culturing, in accordance with various embodiments.

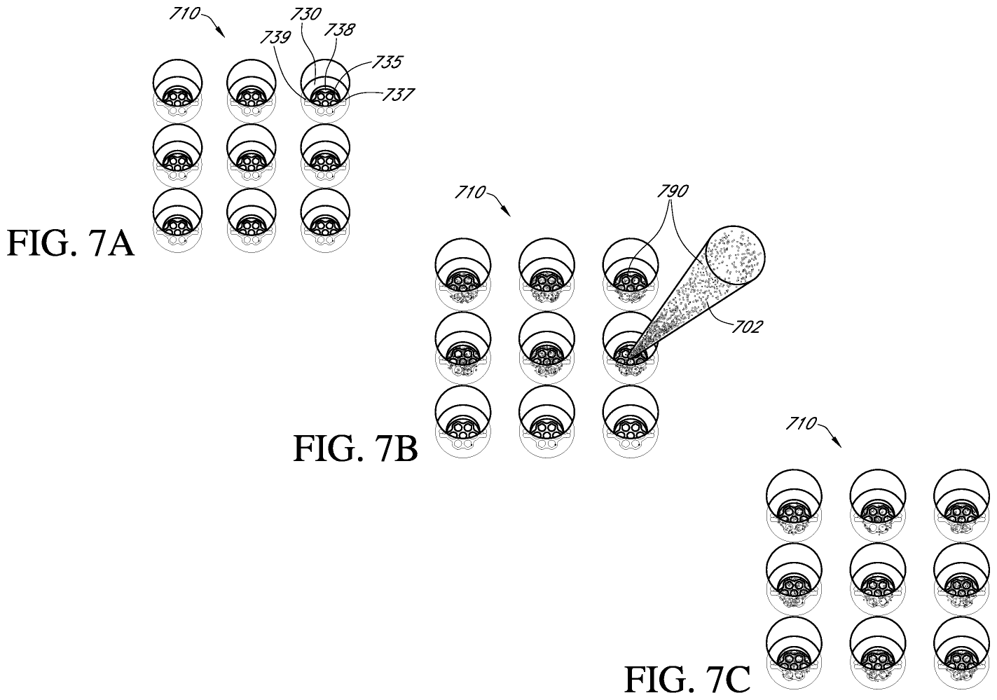

FIGS. 7A, 7B, 7C illustrate various stages of cell loading in an example bio-assembly 710 having an array of bio-scaffolds 730, according to various embodiments. As shown in FIG. 7A, the bio-assembly 710 includes the array of bio-scaffolds 730 where each of the bio-scaffolds 730 includes a vascular component 735 and a void 738. In accordance with various embodiments, each of the vascular components 735 includes a vascular inlet 737 and a vascular outlet 739. In accordance with various embodiments, the bio-assembly 710, the bio-scaffolds 730, the vascular components 735, the vascular inlets 737, the vascular outlets 739, and the voids 738 are similar or identical to the bio-assemblies 110 and/or 210, the bio-scaffolds 130 and/or 230, the vascular components 135 and/or 235, the vascular inlets, the vascular outlets, and the voids as described with respect to FIGS. 1 and 2, and therefore will not be described in further detail. As illustrated in FIG. 7B, various cells or cell types 790 can be disposed in each of the voids 738 of each of the bio-assemblies 710 via the use of a pipet 702. FIG. 7C illustrates the bio-assembly 710 having the various cells or cell types 790 in the voids 738, which can be used for cell culturing, for example, in high-throughput experiments, in accordance with various embodiments.

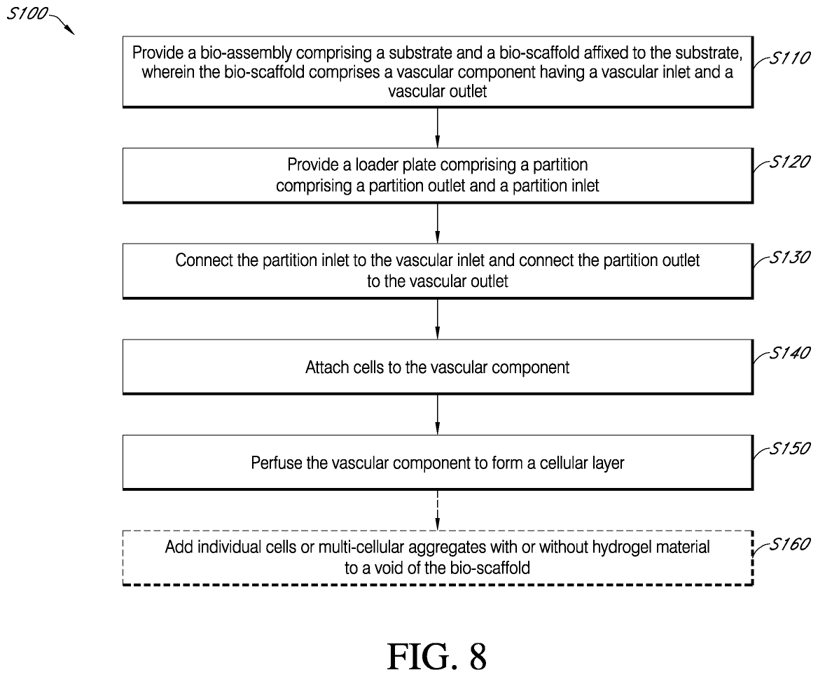

FIG. 8 is a flowchart for a method S100 of generating a cell culture, in accordance with various embodiments. In accordance with various embodiments, the method S100 includes at step S110 providing a bio-assembly comprising a substrate and a bio-scaffold affixed to the substrate. In accordance with various embodiments, the substrate is glass. In accordance with various embodiments, the bio-scaffold is a hydrogel. In accordance with various embodiments, the bio-scaffold comprising the vascular component is 3D printed. In accordance with various embodiments, the bio-assembly is similar to the bio-assemblies 110, 210, and/or 310, and therefore will not be described in further detail.

In accordance with various embodiments, the bio-scaffold includes a vascular component having a vascular inlet and a vascular outlet. In accordance with various embodiments, the vascular component is similar to the vascular components 135, 235, 335, 435a and/or 435b, and therefore will not be described in further detail. In accordance with various embodiments, the vascular inlet is similar to the vascular inlets described with respect to FIGS. 1-7, and therefore will not be described in further detail. In accordance with various embodiments, the vascular outlet is similar to the vascular outlets described with respect to FIGS. 1-7, and therefore will not be described in further detail.

In accordance with various embodiments, the substrate is similar to the substrates 140, 240, and/or 340, and therefore will not be described in further detail. In accordance with various embodiments, the bio-scaffold is similar to the bio-scaffolds 130, 230, and/or 330, and therefore will not be described in further detail.

As shown in FIG. 8, the method S100 includes at step S120 providing a loader plate comprising a partition comprising a partition outlet and a partition inlet. In accordance with various embodiments, the loader plate is similar to the loader plates 120, 320, 420a and/or 420b, and therefore will not be described in further detail. In accordance with various embodiments, the partition inlet is similar to the partition inlets 152, 352, 452a and/or 452b, and therefore will not be described in further detail. In accordance with various embodiments, the partition outlet is similar to the partition outlets 154, 354, 454a and/or 454b, and therefore will not be described in further detail.

As shown in FIG. 8, the method S100 includes at step S130 connecting the partition inlet to the vascular inlet and connecting the partition outlet to the vascular outlet. In accordance with various embodiments, connecting the partition inlet to the vascular inlet and connecting the partition outlet to the vascular outlet occurs when the bio-scaffold is moved into an inner volume of the partition of the loader plate. In accordance with various embodiments, connecting the partition inlet to the vascular inlet and connecting the partition outlet to the vascular outlet occurs in sealed connections with impermeable interfaces between the partition and vascular inlets and outlets.

As shown in FIG. 8, the method S100 includes at step S140 attaching cells to the vascular component. In accordance with various embodiments, the vascular inlet is in fluid communication with the partition inlet. In accordance with various embodiments, the vascular outlet in fluid communication with the partition outlet. In accordance with various embodiments, the vascular component comprises a narrowing inlet and/or a narrowing outlet. In accordance with various embodiments, the vascular component comprises one or more vascular inlets. In accordance with various embodiments, the vascular component includes one or more vascular outlets. In accordance with various embodiments, the bio-scaffold comprises more than one vascular component. In accordance with various embodiments, each of the more than one vascular component comprises a vascular inlet and a vascular outlet. In accordance with various embodiments, the partition inlet and the partition outlet are substantially parallel to a top surface of the loader plate. In accordance with various embodiments, the partition comprises more than one partition inlet and more than one partition outlet, and wherein each of the one or more vascular inlets is in fluid communication with an associated partition inlet and each of the one or more vascular outlets is in fluid communication with an associated partition outlet. In accordance with various embodiments, the bio-scaffold and partition fluid communication is mediated by a tapered constriction in the bio-scaffold and provides a fluidic seal at a normal operating fluid pressure. In accordance with various embodiments, the normal operating fluid pressure is between −100 kPa and 100 kPa. In accordance with various embodiments, the normal operating fluid pressure is between −15 kPa and 15 kPa. In accordance with various embodiments, the normal operating fluid pressure is between −10 kPa and 10 kPa.

In accordance with various embodiments, the method further comprises injecting a fluid mixture into the bio-scaffold, wherein the fluid mixture comprises multiple fluid components. In accordance with various embodiments, the fluid mixture comprises a liquid, foam, or secondary pre-matrix.

In accordance with various embodiments, the bio-scaffold comprises a void. In accordance with various embodiments, the void is a perfusable or injectable space with one or more inlets. In accordance with various embodiments, the void is a perfusable or injectable space with one or more outlets. In accordance with various embodiments, the fluid mixture is configured to be combined with live cells, the combination being injectable into the void. In accordance with various embodiments, the void comprises physical anchors. In accordance with various embodiments, a vascular component is disposed in the void. In accordance with various embodiments, the bio-scaffold is substantially transparent. In accordance with various embodiments, the loader plate comprises a plurality of partitions. In accordance with various embodiments, each of the plurality of partitions comprises a partition inlet and a partition outlet. In accordance with various embodiments, the vascular inlet and vascular outlet for a first vascular component is positioned substantially orthogonal to the vascular inlet and vascular outlet of a second vascular component. In accordance with various embodiments, the loader plate includes 1 to 1000 partitions, including 3, 4, 6, 12, 24, 48, 96, 192, or 384 partitions. In accordance with various embodiments, the partition includes at least one set, and at most 20 sets, of partition inlets and partition outlets.

In accordance with various embodiments, the cells can be any cell or cell types that can form one or more layers from the list consisting of an endothelial layer, an epithelial layer, a smooth muscle cell layer, a sequentially delivered smooth muscle cell layer and endothelial layer, a sequentially delivered smooth muscle cell layer and epithelial layer, a sequentially delivered smooth muscle cell layer, gel layer, endothelial or epithelial layer, a sequentially delivered pericyte layer and endothelial layer, or a sequentially delivered pericyte layer and epithelial layer.

As shown in FIG. 8, the method S100 includes at step S150 perfusing the vascular component to form a cellular layer. In accordance with various embodiments, the cellular layer can include one or more of an endothelial layer, an epithelial layer, a smooth muscle cell layer, a sequentially delivered smooth muscle cell layer and endothelial layer, a sequentially delivered smooth muscle cell layer and epithelial layer, a sequentially delivered smooth muscle cell layer, gel layer, endothelial or epithelial layer, a sequentially delivered pericyte layer and endothelial layer, or a sequentially delivered pericyte layer and epithelial layer.

In accordance with various embodiments, the method S100 optionally includes at step S160 adding individual cells or multi-cellular aggregates with or without hydrogel material to the void of the bio-scaffold.

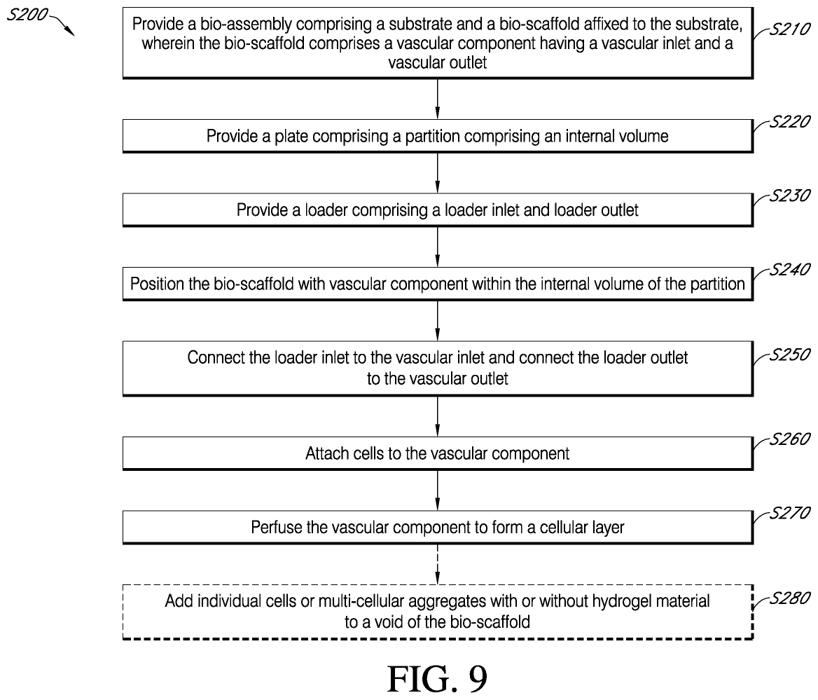

FIG. 9 is another flowchart for a method S200 of generating a cell culture, in accordance with various embodiments. In accordance with various embodiments, the method S200 includes at step S210 providing a bio-assembly comprising a substrate and a bio-scaffold affixed to the substrate. In accordance with various embodiments, the substrate is glass. In accordance with various embodiments, the bio-scaffold is a hydrogel. In accordance with various embodiments, the bio-scaffold comprising the vascular component is 3D printed. In accordance with various embodiments, the bio-assembly is similar to the bio-assemblies 110, 210, and/or 310, and therefore will not be described in further detail. In accordance with various embodiments, the substrate is similar to the substrates 140, 240, and/or 340, and therefore will not be described in further detail. In accordance with various embodiments, the bio-scaffold is similar to the bio-scaffolds 130, 230, and/or 330, and therefore will not be described in further detail.

In accordance with various embodiments, the bio-scaffold includes a vascular component having a vascular inlet and a vascular outlet. In accordance with various embodiments, the vascular component is similar to the vascular components 135, 235, 335, 435a and/or 435b, and therefore will not be described in further detail. In accordance with various embodiments, the vascular inlet is similar to the vascular inlets described with respect to FIGS. 1-7, and therefore will not be described in further detail. In accordance with various embodiments, the vascular outlet is similar to the vascular outlets described with respect to FIGS. 1-7, and therefore will not be described in further detail.

As shown in FIG. 9, the method S200 includes at step S220 providing a plate comprising a partition comprising an internal volume. In accordance with various embodiments, the plate is similar to the plates 220 and/or 520, and therefore will not be described in further detail. In accordance with various embodiments, the internal volume is similar to the internal volumes 255 and/or 355, and therefore will not be described in further detail.

As shown in FIG. 9, the method S200 includes at step S230 providing a loader comprising a loader inlet and loader outlet. In accordance with various embodiments, the loader is similar to the loader 260 and/or 560, and therefore will not be described in further detail. In accordance with various embodiments, the loader inlet is similar to the loader inlets 262 and/or 562, and therefore will not be described in further detail. In accordance with various embodiments, the partition outlet is similar to the loader outlets 264 and/or 564, and therefore will not be described in further detail.

As shown in FIG. 9, the method S200 includes at step S240 positioning the bio-scaffold with vascular component within the internal volume of the partition.

As shown in FIG. 9, the method S200 includes at step S250 connecting the loader inlet to the vascular inlet and connecting the loader outlet to the vascular outlet. In accordance with various embodiments, wherein the vascular inlet is in fluid communication with the loader inlet. In accordance with various embodiments, the vascular outlet in fluid communication with the loader outlet. In accordance with various embodiments, the vascular component comprises a narrowing inlet and/or a narrowing outlet.

In accordance with various embodiments, the vascular component comprises one or more vascular inlets. In accordance with various embodiments, the vascular component includes one or more vascular outlets. In accordance with various embodiments, the bio-scaffold comprises more than one vascular component. In accordance with various embodiments, each of the more than one vascular component comprises a vascular inlet and a vascular outlet.

In accordance with various embodiments, the loader inlet and the loader outlet are substantially orthogonal to a top surface of the plate. In accordance with various embodiments, the loader comprises more than one loader inlet and more than one loader outlet. In accordance with various embodiments, each of the vascular inlets is in fluid communication with an associated loader inlet and each of the vascular outlets is in fluid communication with an associated loader outlet.

In accordance with various embodiments, the bio-scaffold and loader fluid communication is mediated by a tapered constriction in the bio-scaffold and provides a fluidic seal at a normal operating fluid pressure. In accordance with various embodiments, the normal operating fluid pressure is between −100 kPa and 100 kPa. In accordance with various embodiments, the normal operating fluid pressure is between −15 kPa and 15 kPa. In accordance with various embodiments, the normal operating fluid pressure is between −10 kPa and 10 kPa.

In accordance with various embodiments, the method further includes injecting a fluid mixture into the bio-scaffold, wherein the fluid mixture comprises multiple fluid components. In accordance with various embodiments, the fluid mixture comprises a liquid, foam, or secondary pre-matrix.

In accordance with various embodiments, the bio-scaffold comprises a void, wherein a vascular component is disposed in the void. In accordance with various embodiments, the void is a perfusable or injectable space with one or more inlets. In accordance with various embodiments, the void is a perfusable or injectable space with one or more outlets. In accordance with various embodiments, the fluid mixture is configured to be combined with live cells, the combination being injectable into the void. In accordance with various embodiments, the void comprises physical anchors. In accordance with various embodiments, the bio-scaffold is substantially transparent. In accordance with various embodiments, the bio-scaffold further comprises a hydrophilic component and a hydrophobic component.

In accordance with various embodiments, the plate comprises a plurality of partitions. In accordance with various embodiments, at least one loader inlet and at least one loader outlet in associated with each of the plurality of partitions. In accordance with various embodiments, the vascular inlet and vascular outlet for a first vascular component is positioned substantially orthogonal to the vascular inlet and vascular outlet of a second vascular component. In accordance with various embodiments, the plate comprises 1 to 1000 partitions, including 1 to 1000 partitions, including 3, 4, 6, 12, 24, 48, 96, 192, or 384 partitions. In accordance with various embodiments, the loader comprises up to 384 sets of loader inlets and loader outlets.

In accordance with various embodiments, the loader further includes a fluid inlet channel and a fluid outlet channel. In accordance with various embodiments, the fluid inlet channel is in fluid communication with the loader inlet. In accordance with various embodiments, the fluid outlet channel is in fluid communication with the loader outlet. In accordance with various embodiments, the fluid inlet channel is in fluid communication with more than one loader inlet. In accordance with various embodiments, the fluid outlet channel is in fluid communication with more than one loader outlet.

In accordance with various embodiments, the fluid outlet of one loader serves as the fluid inlet of one or more alternate loaders on the same device. In accordance with various embodiments, the fluid outlet of one loader servers as the fluid inlet of one or more alternate loaders on a different device. In accordance with various embodiments, the fluid outlet of one loader servers as the fluid inlet of one or more alternate loaders on the same device and/or on different devices.

As shown in FIG. 9, the method S200 includes at step S260 attaching cells to the vascular component. In accordance with various embodiments, the cells can be any cell or cell types that can form one or more layers from the list consisting of an endothelial layer, an epithelial layer, a smooth muscle cell layer, a sequentially delivered smooth muscle cell layer and endothelial layer, a sequentially delivered smooth muscle cell layer and epithelial layer, a sequentially delivered smooth muscle cell layer, gel layer, endothelial or epithelial layer, a sequentially delivered pericyte layer and endothelial layer, or a sequentially delivered pericyte layer and epithelial layer.

As shown in FIG. 9, the method S200 includes at step S270 perfusing the vascular component to form a cellular layer. In accordance with various embodiments, the cellular layer can include one or more of an endothelial layer, an epithelial layer, a smooth muscle cell layer, a sequentially delivered smooth muscle cell layer and endothelial layer, a sequentially delivered smooth muscle cell layer and epithelial layer, a sequentially delivered smooth muscle cell layer, gel layer, endothelial or epithelial layer, a sequentially delivered pericyte layer and endothelial layer, or a sequentially delivered pericyte layer and epithelial layer.

In accordance with various embodiments, the method S200 optionally includes at step S280 adding individual cells or multi-cellular aggregates with or without hydrogel material to the void of the bio-scaffold.

### Recitation of Embodiments

Embodiment 1: A kit comprising a bio-assembly comprising a substrate, and a bio-scaffold affixed to the substrate; and a loader plate comprising a partition comprising a partition outlet and a partition inlet, the partition outlet and the partition inlet in fluid communication with the bio-scaffold, and a bio-compatible adhesive positioned between the substrate and the loader plate, the adhesive configured to maintain a fluid-impermeable bond between the substrate and the loader plate.

Embodiment 2: The kit of Embodiment 1, further comprising a fluid mixture configured to be injected into the bio-scaffold.

Embodiment 3: The kit of any preceding Embodiment, wherein the substrate is glass, wherein the bio-scaffold is covalently bonded to the substrate.

Embodiment 4: The kit of any preceding Embodiment, wherein the bio-scaffold is a hydrogel.

Embodiment 5: The kit of any preceding Embodiment, wherein the bio-scaffold is 3D printed.

Embodiment 6: The kit of any preceding Embodiment, wherein the bio-scaffold comprises a vascular component having a vascular inlet and a vascular outlet.

Embodiment 7: The kit of Embodiment 6, wherein the vascular inlet is in fluid communication with the partition inlet.

Embodiment 8: The kit of Embodiment 7, wherein the vascular outlet is in fluid communication with the partition outlet.

Embodiment 9: The kit of Embodiment 6, wherein the vascular component comprises a narrowing inlet and/or a narrowing outlet.

Embodiment 10: The kit of Embodiment 6, wherein the vascular component comprises one or more vascular inlets.

Embodiment 11: The kit of Embodiment 10, wherein the vascular component includes one or more vascular outlets.

Embodiment 12: The kit of Embodiment 6, wherein the bio-scaffold comprises more than one vascular component.

Embodiment 13: The kit of Embodiment 12, wherein each of the more than one vascular component comprises a vascular inlet and a vascular outlet.

Embodiment 14: The kit of any preceding Embodiment, wherein the partition inlet and the partition outlet are substantially parallel to a top surface of the loader plate.

Embodiment 15: The kit of Embodiment 11, wherein the partition comprises more than one partition inlet and more than one partition outlet, and wherein each of the one or more vascular inlets is in fluid communication with an associated partition inlet and each of the one or more vascular outlets is in fluid communication with an associated partition outlet.

Embodiment 16: The kit of any preceding Embodiment, wherein the bio-scaffold and partition fluid communication are mediated by a tapered constriction in the bio-scaffold and provides a fluidic seal at a normal operating fluid pressure.

Embodiment 17: The kit of Embodiment 16, wherein the normal operating fluid pressure is between −100 kPa and 100 kPa.

Embodiment 18: The kit of Embodiment 16, wherein the normal operating fluid pressure is between −15 kPa and 15 kPa.

Embodiment 19: The kit of any preceding Embodiment 16, wherein the normal operating fluid pressure is between −10 kPa and 10 kPa.

Embodiment 20: The kit of Embodiment 2, wherein the fluid mixture comprises multiple fluid components.

Embodiment 21: The kit of Embodiment 2, wherein the fluid mixture comprises a liquid, foam, or secondary pre-matrix.

Embodiment 22: The kit of Embodiment 2, wherein the bio-scaffold comprises a void.

Embodiment 23: The kit of Embodiment 22, wherein the void is a perfusable or injectable space with one or more inlets.

Embodiment 24: The kit of Embodiment 23, wherein the void is a perfusable or injectable space with one or more outlets.

Embodiment 25: The kit of Embodiment 22, wherein the fluid mixture is configured to be combined with live cells, the combination being injectable into the void.

Embodiment 26: The kit of Embodiment 22, wherein the void comprises physical anchors.

Embodiment 27: The kit of Embodiment 22, wherein a vascular component is disposed in the void.

Embodiment 28: The kit of any preceding Embodiment, wherein the bio-scaffold is substantially transparent.

Embodiment 29: The kit of any preceding Embodiment, wherein the bio-scaffold further comprises a hydrophilic component and a hydrophobic component.

Embodiment 30: The kit of any preceding Embodiment, wherein the loader plate comprises a plurality of partitions.

Embodiment 31: The kit of Embodiment 30, wherein each of the plurality of partitions comprises a partition inlet and a partition outlet.

Embodiment 32: The kit of Embodiment 12, wherein the vascular inlet and vascular outlet for a first vascular component is positioned substantially orthogonal to the vascular inlet and vascular outlet of a second vascular component.

Embodiment 33: The kit of any preceding Embodiment, wherein the loader plate comprises between 3 and 384 partitions.

Embodiment 34: The kit of any preceding Embodiment, wherein the partition comprises up to 384 sets of partition inlets and partition outlets.

Embodiment 35: A kit comprising a bio-assembly comprising a substrate, and bio-scaffold affixed to the substrate; plate comprising partition comprising an internal volume and shaped to receive the bio-scaffold into the internal volume, and bio-compatible adhesive positioned between the substrate and the plate, the adhesive configured to maintain a bond between the substrate and the plate; loader comprising a loader inlet and a loader outlet, the loader inlet and loader outlet in fluid communication with the bio-scaffold; and a fluid mixture configured to be injected into the bio-scaffold.

Embodiment 36: The kit of Embodiment 35, wherein the substrate is glass, wherein the bio-scaffold is covalently bonded to the substrate.

Embodiment 37: The kit of any preceding Embodiment, wherein the bio-scaffold is a hydrogel.

Embodiment 38: The kit of any preceding Embodiment, wherein the bio-scaffold comprises a vascular component.

Embodiment 39: The kit of Embodiment 38, wherein the bio-scaffold comprising the vascular component is 3D printed.

Embodiment 40: The kit of Embodiment 38, wherein the vascular component comprises a vascular inlet and a vascular outlet.

Embodiment 41: The kit of Embodiment 40, wherein the vascular inlet is in fluid communication with the loader inlet.

Embodiment 42: The kit of Embodiment 41, wherein the vascular outlet in fluid communication with the loader outlet.

Embodiment 43: The kit of Embodiment 38, wherein the vascular component comprises a narrowing inlet and/or a narrowing outlet.

Embodiment 44: The kit of Embodiment 38, wherein the vascular component comprises one or more vascular inlets.

Embodiment 45: The kit of Embodiment 44, wherein the vascular component includes one or more vascular outlets.

Embodiment 46: The kit of any preceding Embodiment, wherein the bio-scaffold comprises more than one vascular component.

Embodiment 47: The kit of Embodiment 46, wherein each of the more than one vascular component comprises a vascular inlet and a vascular outlet.

Embodiment 48: The kit of any preceding Embodiment, wherein the loader inlet and the loader outlet are substantially orthogonal to a top surface of the plate.

Embodiment 49: The kit of Embodiment 47, wherein the loader comprises more than one loader inlet and more than one loader outlet, and wherein each of the vascular inlets is in fluid communication with an associated loader inlet and each of the vascular outlets is in fluid communication with an associated loader outlet.

Embodiment 50: The kit of any preceding Embodiment, wherein the bio-scaffold and loader fluid communication are mediated by a tapered constriction in the bio-scaffold and provides a fluidic seal at a normal operating fluid pressure.

Embodiment 51: The kit of Embodiment 50, wherein the normal operating fluid pressure is between −100 kPa and 100 kPa.

Embodiment 52: The kit of Embodiment 50, wherein the normal operating fluid pressure is between −15 kPa and 15 kPa.

Embodiment 53: The kit of Embodiment 50, wherein the normal operating fluid pressure is between −10 kPa and 10 kPa.

Embodiment 54: The kit of any preceding Embodiment, wherein the fluid mixture comprises multiple fluid components.

Embodiment 55: The kit of any preceding Embodiment, wherein the fluid mixture comprises a liquid, foam, or secondary pre-matrix.

Embodiment 56: The kit of any preceding Embodiment, wherein the bio-scaffold comprises a void, wherein a vascular component is disposed in the void.

Embodiment 57: The kit of Embodiment 56, wherein the void is a perfusable or injectable space with one or more inlets.

Embodiment 58: The kit of Embodiment 57, wherein the void is a perfusable or injectable space with one or more outlets.

Embodiment 59: The kit of Embodiment 56, wherein the fluid mixture is configured to be combined with live cells, the combination being injectable into the void.

Embodiment 60: The kit of Embodiment 56, wherein the void comprises physical anchors.

Embodiment 61: The kit of any preceding Embodiment, wherein the bio-scaffold is substantially transparent.

Embodiment 62: The kit of any preceding Embodiment, wherein the bio-scaffold further comprises a hydrophilic component and a hydrophobic component.

Embodiment 63: The kit of any preceding Embodiment, wherein the plate comprises a plurality of partitions.

Embodiment 64: The kit of Embodiment 63, wherein at least one loader inlet and at least one loader outlet in associated with each of the plurality of partitions.

Embodiment 65: The kit of Embodiment 45, wherein the vascular inlet and vascular outlet for a first vascular component is positioned substantially orthogonal to the vascular inlet and vascular outlet of a second vascular component.

Embodiment 66: The kit of any preceding Embodiment, wherein the loader comprises between 3 and 384 partitions.

Embodiment 67: The kit of any preceding Embodiment, wherein the loader comprises up to 384 sets of loader inlets and loader outlets.

Embodiment 68: The kit of any preceding Embodiment, wherein the loader further comprising a fluid inlet channel and a fluid outlet channel.

Embodiment 69: The kit of Embodiment 68, wherein the fluid inlet channel is in fluid communication with the loader inlet.

Embodiment 70: The kit of Embodiment 69, wherein the fluid outlet channel is in fluid communication with the loader outlet.

Embodiment 71: The kit of Embodiment 68, wherein the fluid inlet channel is in fluid communication with more than one loader inlet.

Embodiment 72: The kit of Embodiment 68, wherein the fluid outlet channel is in fluid communication with more than one loader outlet.

Embodiment 73: The kit of Embodiment 68, wherein the fluid outlet of one loader serves as the fluid inlet of one or more alternate loaders on the same device.

Embodiment 74: The kit of Embodiment 68, wherein the fluid outlet of one loader servers as the fluid inlet of one or more alternate loaders on a different device.

Embodiment 75: The kit of Embodiment 68, wherein the fluid outlet of one loader servers as the fluid inlet of one or more alternate loaders on the same device and/or on different devices.

Embodiment 76: A method for generating a kit containing cells, the method comprising providing a bio-assembly comprising a substrate and a bio-scaffold affixed to the substrate, wherein the bio-scaffold comprises a vascular component having a vascular inlet and a vascular outlet; providing a loader plate comprising a partition comprising a partition outlet and a partition inlet; connecting the partition inlet to the vascular inlet and connecting the partition outlet to the vascular outlet; attaching cells to the vascular component; and perfusing the vascular component to form a cellular layer.

Embodiment 77: The method of Embodiment 76, wherein the cellular layer comprises an endothelial layer.

Embodiment 78: The method of any preceding Embodiment, wherein the cellular layer comprises an epithelial layer.

Embodiment 79: The method of any preceding Embodiment, wherein the cellular layer comprises a smooth muscle cell layer.

Embodiment 80: The method of any preceding Embodiment, wherein the cellular layer comprises a sequentially delivered smooth muscle cell layer and endothelial layer.

Embodiment 81: The method of any preceding Embodiment, wherein the cellular layer comprises a sequentially delivered smooth muscle cell layer and epithelial layer.

Embodiment 82: The method of any preceding Embodiment, wherein the cellular layer comprises a sequentially delivered smooth muscle cell layer, gel layer, endothelial or epithelial layer.

Embodiment 83: The method of any preceding Embodiment, wherein the cellular layer comprises a sequentially delivered pericyte layer and endothelial layer.

Embodiment 84: The method of any preceding Embodiment, wherein the cellular layer comprises a sequentially delivered pericyte layer and epithelial layer.

Embodiment 85: The method of any preceding Embodiment, wherein the substrate is glass.

Embodiment 86: The method of any preceding Embodiment, wherein the bio-scaffold is a hydrogel.

Embodiment 87: The method of any preceding Embodiment, wherein the bio-scaffold comprises a vascular component.

Embodiment 88: The method of Embodiment 87, wherein the bio-scaffold comprising the vascular component is 3D printed.

Embodiment 89: The method of Embodiment 87, wherein the vascular component comprises a vascular inlet and a vascular outlet.

Embodiment 90: The method of Embodiment 89, wherein the vascular inlet is in fluid communication with the partition inlet.

Embodiment 91: The method of Embodiment 90, wherein the vascular outlet in fluid communication with the partition outlet.

Embodiment 92: The method of Embodiment 87, wherein the vascular component comprises a narrowing inlet and/or a narrowing outlet.

Embodiment 93: The method of Embodiment 87, wherein the vascular component comprises one or more vascular inlets.

Embodiment 94: The method of Embodiment 93, wherein the vascular component includes one or more vascular outlets.

Embodiment 95: The method of any preceding Embodiment, wherein the bio-scaffold comprises more than one vascular component.

Embodiment 96: The method of Embodiment 95, wherein each of the more than one vascular component comprises a vascular inlet and a vascular outlet.

Embodiment 97: The method of any preceding Embodiment, wherein the partition inlet and the partition outlet are substantially parallel to a top surface of the loader plate.

Embodiment 98: The method of Embodiment 94, wherein the partition comprises more than one partition inlet and more than one partition outlet, and wherein each of the one or more vascular inlets is in fluid communication with an associated partition inlet and each of the one or more vascular outlets is in fluid communication with an associated partition outlet.

Embodiment 99: The method of any preceding Embodiment, wherein the bio-scaffold and partition fluid communication is mediated by a tapered constriction in the bio-scaffold and provides a fluidic seal at a normal operating fluid pressure.

Embodiment 100: The method of Embodiment 99, wherein the normal operating fluid pressure is between −100 kPa and 100 kPa.

Embodiment 101: The method of Embodiment 99, wherein the normal operating fluid pressure is between −15 kPa and 15 kPa.

Embodiment 102: The method of Embodiment 99, wherein the normal operating fluid pressure is between −10 kPa and 10 kPa.

Embodiment 103: The method of any preceding Embodiment, further comprising injecting a fluid mixture into the bio-scaffold, wherein the fluid mixture comprises multiple fluid components.

Embodiment 104: The method of any preceding Embodiment, wherein the fluid mixture comprises a liquid, foam, or secondary pre-matrix.

Embodiment 105: The method of any preceding Embodiment, wherein the bio-scaffold comprises a void.

Embodiment 106: The method of Embodiment 105, wherein the void is a perfusable or injectable space with one or more inlets.

Embodiment 107: The method of Embodiment 106, wherein the void is a perfusable or injectable space with one or more outlets.

Embodiment 108: The method of Embodiment 105, wherein the fluid mixture is configured to be combined with live cells, the combination being injectable into the void.

Embodiment 109: The method of Embodiment 105, wherein the void comprises physical anchors.

Embodiment 110: The method of Embodiment 105, wherein a vascular component is disposed in the void.

Embodiment 111: The method of any preceding Embodiment, wherein the bio-scaffold is substantially transparent.

Embodiment 112: The method of any preceding Embodiment, wherein the bio-scaffold further comprises a hydrophilic component and a hydrophobic component.

Embodiment 113: The method of any preceding Embodiment, wherein the loader plate comprises a plurality of partitions.

Embodiment 114: The method of Embodiment 113, wherein each of the plurality of partitions comprises a partition inlet and a partition outlet.

Embodiment 115: The method of any preceding Embodiment 95, wherein the vascular inlet and vascular outlet for a first vascular component is positioned substantially orthogonal to the vascular inlet and vascular outlet of a second vascular component.

Embodiment 116: The method of any preceding Embodiment, wherein the loader plate comprises between 3 and 384 partitions.

Embodiment 117: The method of any preceding Embodiment, wherein the partition comprises up to 20 sets of partition inlets and partition outlets.

Embodiment 118: A method for generating a cell culture, the method comprising providing a bio-assembly comprising a substrate and a bio-scaffold affixed to the substrate, wherein the bio-scaffold comprises a vascular component having a vascular inlet and a vascular outlet; providing a plate comprising a partition comprising an internal volume; providing a loader comprising a loader inlet and loader outlet; positioning the bio-scaffold with vascular component within the internal volume of the partition; connecting the loader inlet to the vascular inlet and connecting the loader outlet to the vascular outlet; attaching cells to the vascular component; and perfusing the vascular component to form a cellular layer.

Embodiment 119: The method of Embodiment 118, wherein the cellular layer comprises an endothelial layer.

Embodiment 120: The method of any preceding Embodiment, wherein the cellular layer comprises an epithelial layer.

Embodiment 121: The method of any preceding Embodiment, wherein the cellular layer comprises a smooth muscle cell layer.

Embodiment 122: The method of any preceding Embodiment, wherein the cellular layer comprises a sequentially delivered smooth muscle cell layer and endothelial layer.

Embodiment 123: The method of any preceding Embodiment, wherein the cellular layer comprises a sequentially delivered smooth muscle cell layer and epithelial layer.

Embodiment 124: The method of any preceding Embodiment, wherein the cellular layer comprises a sequentially delivered smooth muscle cell layer, gel layer, endothelial or epithelial layer.

Embodiment 125: The method of any preceding Embodiment, wherein the cellular layer comprises a sequentially delivered pericyte layer and endothelial layer.

Embodiment 126: The method of any preceding Embodiment, wherein the cellular layer comprises a sequentially delivered pericyte layer and epithelial layer.

Embodiment 127: The method of any preceding Embodiment, wherein the substrate is glass.

Embodiment 128: The method of any preceding Embodiment, wherein the bio-scaffold is a hydrogel.

Embodiment 129: The method of any preceding Embodiment, wherein the bio-scaffold comprises a vascular component.

Embodiment 130: The method of Embodiment 129, wherein the bio-scaffold comprising the vascular component is 3D printed.

Embodiment 131: The method of Embodiment 129, wherein the vascular component comprises a vascular inlet and a vascular outlet.

Embodiment 132: The method of Embodiment 131, wherein the vascular inlet is in fluid communication with the loader inlet.

Embodiment 133: The method of Embodiment 132, wherein the vascular outlet in fluid communication with the loader outlet.

Embodiment 134: The method of Embodiment 129, wherein the vascular component comprises a narrowing inlet and/or a narrowing outlet.

Embodiment 135: The method of Embodiment 129, wherein the vascular component comprises one or more vascular inlets.

Embodiment 136: The method of Embodiment 135, wherein the vascular component includes one or more vascular outlets.

Embodiment 137: The method of any preceding Embodiment, wherein the bio-scaffold comprises more than one vascular component.

Embodiment 138: The method of Embodiment 137, wherein each of the more than one vascular component comprises a vascular inlet and a vascular outlet.

Embodiment 139: The method of any preceding Embodiment, wherein the loader inlet and the loader outlet are substantially orthogonal to a top surface of the plate.

Embodiment 140: The method of Embodiment 138, wherein the loader comprises more than one loader inlet and more than one loader outlet, and wherein each of the vascular inlets is in fluid communication with an associated loader inlet and each of the vascular outlets is in fluid communication with an associated loader outlet.

Embodiment 141: The method of any preceding Embodiment, wherein the bio-scaffold and loader fluid communication is mediated by a tapered constriction in the bio-scaffold and provides a fluidic seal at a normal operating fluid pressure.

Embodiment 142: The method of Embodiment 141, wherein the normal operating fluid pressure is between −100 kPa and 100 kPa.

Embodiment 143: The method of Embodiment 141, wherein the normal operating fluid pressure is between −15 kPa and 15 kPa.

Embodiment 144: The method of Embodiment 141, wherein the normal operating fluid pressure is between −10 kPa and 10 kPa.

Embodiment 145: The method of any preceding Embodiment, further comprising injecting a fluid mixture into the bio-scaffold, wherein the fluid mixture comprises multiple fluid components.

Embodiment 146: The method of Embodiment 145, wherein the fluid mixture comprises a liquid, foam, or secondary pre-matrix.

Embodiment 147: The method of any preceding Embodiment, wherein the bio-scaffold comprises a void, wherein a vascular component is disposed in the void.

Embodiment 148: The method of Embodiment 147, wherein the void is a perfusable or injectable space with one or more inlets.

Embodiment 149: The method of Embodiment 148, wherein the void is a perfusable or injectable space with one or more outlets.

Embodiment 150: The method of Embodiment 147, wherein the fluid mixture is configured to be combined with live cells, the combination being injectable into the void.

Embodiment 151: The method of Embodiment 147, wherein the void comprises physical anchors.

Embodiment 152: The method of any preceding Embodiment, wherein the bio-scaffold is substantially transparent.

Embodiment 153: The method of any preceding Embodiment, wherein the bio-scaffold further comprises a hydrophilic component and a hydrophobic component.

Embodiment 154: The method of any preceding Embodiment, wherein the plate comprises a plurality of partitions.

Embodiment 155: The method of Embodiment 154, wherein at least one loader inlet and at least one loader outlet in associated with each of the plurality of partitions.

Embodiment 156: The method of Embodiment 136, wherein the vascular inlet and vascular outlet for a first vascular component is positioned substantially orthogonal to the vascular inlet and vascular outlet of a second vascular component.

Embodiment 157: The method of any preceding Embodiment, wherein the loader plate comprises between 3 and 384 partitions.

Embodiment 158: The method of any preceding Embodiment, wherein the loader comprises up to 384 sets of loader inlets and loader outlets.

Embodiment 159: The method of any preceding Embodiment, wherein the loader further comprising a fluid inlet channel and a fluid outlet channel.

Embodiment 160: The method of Embodiment 159, wherein the fluid inlet channel is in fluid communication with the loader inlet.

Embodiment 161: The method of Embodiment 160, wherein the fluid outlet channel is in fluid communication with the loader outlet.

Embodiment 162: The method of Embodiment 159, wherein the fluid inlet channel is in fluid communication with more than one loader inlet.

Embodiment 163: The method of Embodiment 159, wherein the fluid outlet channel is in fluid communication with more than one loader outlet.

Embodiment 164: The method of Embodiment 159, wherein the fluid outlet of one loader serves as the fluid inlet of one or more alternate loaders on the same device.

Embodiment 165: The method of Embodiment 159, wherein the fluid outlet of one loader servers as the fluid inlet of one or more alternate loaders on a different device.

Embodiment 166: The method of Embodiment 159, wherein the fluid outlet of one loader servers as the fluid inlet of one or more alternate loaders on the same device and/or on different devices.

Embodiment 167: The kit of Embodiment 33, wherein the loader plate comprises 3, 4, 6, 12, 24, 48, 96, 192, or 384 partitions.

Embodiment 168: The kit of Embodiment 66, wherein the loader plate comprises 3, 4, 6, 12, 24, 48, 96, 192, or 384 partitions.

Embodiment 169: The method of Embodiment 116, wherein the loader plate comprises 3, 4, 6, 12, 24, 48, 96, 192, or 384 partitions.

Embodiment 170: The method of Embodiment 157, wherein the loader plate comprises 3, 4, 6, 12, 24, 48, 96, 192, or 384 partitions.

Embodiment 171: The method of Embodiment 76, further comprising adding individual cells or multi-cellular aggregates with or without hydrogel material to a void of the bio-scaffold.

Embodiment 172: The method of Embodiment 118, further comprising adding individual cells or multi-cellular aggregates with or without hydrogel material to a void of the bio-scaffold.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A kit comprising:
a bio-assembly comprising:
a substrate, and
a bio-scaffold affixed to the substrate; and
a loader plate comprising:
a partition comprising a partition outlet and a partition inlet, the partition outlet and the partition inlet in fluid communication with the bio-scaffold, and
a bio-compatible adhesive positioned between the substrate and the loader plate, the adhesive configured to maintain a fluid-impermeable bond between the substrate and the loader plate.

2. The kit of claim 1, further comprising a fluid mixture configured to be injected into the bio-scaffold.

3. The kit of claim 1, wherein the bio-scaffold is a hydrogel or a vascular component having a vascular inlet and a vascular outlet.

4. The kit of claim 3, wherein the vascular inlet is in fluid communication with the partition inlet and the vascular outlet is in fluid communication with the partition outlet.

5. The kit of claim 3, wherein the vascular component comprises one or more vascular inlets and one or more vascular outlets.

6. The kit of claim 1, wherein the partition inlet and the partition outlet are substantially parallel to a top surface of the loader plate.

7. The kit of claim 6, wherein the partition comprises more than one partition inlet and more than one partition outlet, and wherein each of the one or more vascular inlets is in fluid communication with an associated partition inlet and each of the one or more vascular outlets is in fluid communication with an associated partition outlet.

8. The kit of claim 1, wherein the bio-scaffold and partition fluid communication are mediated by a tapered constriction in the bio-scaffold and provides a fluidic seal at a normal operating fluid pressure.

9. The kit of claim 2, wherein the bio-scaffold comprises a void, the void comprising a perfusable or injectable space with one or more inlets and one or more outlets.

10. The kit of claim 9, wherein the fluid mixture is configured to be combined with live cells, the combination being injectable into the void.

11. A method for generating a kit containing cells, the method comprising:
providing a bio-assembly comprising a substrate and a bio-scaffold affixed to the substrate, wherein the bio-scaffold comprises a vascular component having a vascular inlet and a vascular outlet;
providing a loader plate comprising a partition comprising a partition outlet and a partition inlet;
connecting the partition inlet to the vascular inlet and connecting the partition outlet to the vascular outlet;
attaching cells to the vascular component; and
perfusing the vascular component to form a cellular layer.

12. The method of claim 11, wherein the cellular layer comprises at least one of an endothelial layer, an epithelial layer, a smooth muscle cell layer, a sequentially delivered smooth muscle cell layer and endothelial layer, a sequentially delivered smooth muscle cell layer and epithelial layer, a sequentially delivered smooth muscle cell layer, gel layer, endothelial or epithelial layer, a sequentially delivered pericyte layer and endothelial layer, or a sequentially delivered pericyte layer and epithelial layer.

13. The method of claim 11, further comprising:
injecting a fluid mixture into the bio-scaffold, wherein the fluid mixture comprises multiple fluid components from a list of a liquid, foam, or secondary pre-matrix.

14. The method of claim 11, wherein the bio-scaffold comprises a void, the void comprising a perfusable or injectable space with one or more inlets and one or more outlets.

15. The method of claim 11, further comprising:
adding individual cells or multi-cellular aggregates with or without hydrogel material to a void of the bio-scaffold.

16. A kit comprising:
a bio-assembly comprising:
a substrate, and
a bio-scaffold affixed to the substrate;
a plate comprising:
a partition comprising an internal volume and shaped to receive the bio-scaffold into the internal volume, and
a bio-compatible adhesive positioned between the substrate and the plate, the adhesive configured to maintain a bond between the substrate and the plate;
a loader comprising a loader inlet and a loader outlet, the loader inlet and loader outlet in fluid communication with the bio-scaffold; and
a fluid mixture configured to be injected into the bio-scaffold.

17. The kit of claim 16, wherein the bio-scaffold comprises a vascular component having one or more vascular inlets and one or more vascular outlets.

18. The kit of claim 16, wherein the bio-scaffold and loader fluid communication are mediated by a tapered constriction in the bio-scaffold and provides a fluidic seal at a normal operating fluid pressure.

19. The kit of claim 16, wherein the bio-scaffold comprises a void, wherein a vascular component is disposed in the void.

20. The kit of claim 16, wherein the loader further comprising a fluid inlet channel in fluid communication with the loader inlet and a fluid outlet channel in fluid communication with the loader outlet.
